(19) United States
(12) Patent Application Publication
(10) Pub. No.: US 2013/0303923 A1
(43) Pub. Date: Nov. 14, 2013

(54) SYSTEM AND METHOD FOR VASCULAR TESTING

(71) Applicant: BIOMEDIX, INC., St. Paul, MN (US)

(72) Inventors: David A. Lerner, St. Paul, MN (US); Linda Marie Doran, St. Paul, MN (US)

(73) Assignee: BIOMEDIX, INC., St. Paul, MN (US)

(21) Appl. No.: 13/840,964

(22) Filed: Mar. 15, 2013

Related U.S. Application Data
(60) Provisional application No. 61/646,001, filed on May 11, 2012.

(51) Int. Cl. A61B 5/022

(52) U.S. Cl. CPC A61B 5/02208
USPC 600/492; 600/490

(57) ABSTRACT

A vascular testing system includes a first pressure cuff assembly positionable about a limb of a patient and a controller. The first pressure cuff assembly includes a pressure bladder and a first array of acoustic sensors, the first array including a plurality of acoustic sensor elements arranged circumferentially relative to the pressure bladder. The controller is configured to concurrently sense vascular data at two or more discrete testing locations utilizing at least two different sets of the acoustic sensors of the first array each positioned at or near one of the discrete testing locations. Each set of the acoustic sensors includes one or more of the plurality of acoustic sensor elements.

### CROSS-REFERENCE TO RELATED APPLICATION(S)

The present application claims priority to U.S. Provisional Patent Application Ser. No. 61/646,001, entitled “Noninvasive Sensing System for Vessel-Specific Vascular Testing,” filed May 11, 2012, which is hereby incorporated by reference in its entirety.

### BACKGROUND

The present invention relates to vascular testing, and more particularly to a blood pressure testing (sphygmomanometry) system and method.

In order to assess peripheral arterial vascular flow, it is desirable to assess limb pressure in individual blood vessels rather than solely in the limb as a whole. Existing sphygmomanometry technology uses a Doppler Ultrasound device as a distal return-of-flow sensor. Examples of Doppler-based systems are found in U.S. Pat. Nos. 4,154,238 and 6,740,042. As per “Medical Instruments and Devices” in MEDICAL DEVICES AND SYSTEMS (CRC 2006) (Wolf W. von Maltzahn, University of Texas at Arlington, ed.), the origin of Korotkoff sounds is not seen to be flow turbulence. Rather, it is known that Korotkoff sounds correlate in time (during the cuff deflation and cardiac cycles) with blood velocity sounds produced by Doppler ultrasound waves at a testing location upon deflation of an occlusive cuff. In general, a Doppler ultrasound sensor/probe is located by a clinician proximate to a blood vessel under test (VUT). Locating these VUTs can be challenging, particularly with patients having compromised arterial flow. For instance, one must appreciate surface anatomy to know where to place the Doppler ultrasound probe in order to insonate (expose to ultrasound) a selected VUT properly. Given the limited beam width and focus of diagnostic ultrasound beams, the clinician must place the Doppler ultrasound probe quite close to the location of the blood vessel (as reflected to the surface of the skin) in order to sense and appreciate a blood flow signal. Furthermore, one must also apply acoustic coupling gel to provide for optimal ultrasound transmission and impedance matching.

Alternate sphygmomanometry approaches, to Doppler ultrasound, use oscillometry to measure limb arterial pressure. Oscillometry involves the controlled inflation and deflation of a pressure cuff, with measurement of pressure at the cuff and subsequent analysis of pressure measurements. Examples of oscillometric systems are found in U.S. Pat. Nos. 7,166,076, 7,172,555, and 7,214,192. The oscillometric method assesses limb pressure as a whole without focus on an individual vessel, because the pressure produced by an oscillometric test is reflective of the vessel with the highest pressure in the limb under test rather than in a specific blood vessel (i.e., artery). In oscillometry, an arterial pulse waveform from an applied blood pressure cuff is interrogated while the air pressure in the cuff is deflated from a super-systolic pressure to near zero. A maximum amplitude point has been empirically determined to be mean arterial pressure (MAP), which can be given by equation 1:

$$MAP = \frac{SP + 2 * DP}{3} \qquad ( 1 )$$

where SP is systolic pressure and DP is diastolic pressure. Statistical comparisons of the measured oscillometric pulse waveform with independently measured blood pressures (e.g., using other measurement techniques) can provide a correlation with actual systolic pressure and diastolic pressure. Thus, oscillometry is a technique that is dependent on statistical analyses to correlate the arterial pulse waveform amplitude with systolic and diastolic pressures. Further, the statistical factors must be derived separately for each limb segment. In other words, the empirically-derived mathematical formulae are different for leg versus arm, thigh versus, calf, etc.

Auscultatory devices are also known for blood pressure testing. For example, U.S. Pat. Nos. 4,116,230, 5,680,868 and 5,873,836 disclose electronic auscultatory blood pressure devices, which use microphones in conjunction with a pressure cuff for blood pressure testing. However, such prior art systems are either not vessel-specific (e.g., U.S. Pat. No. 4,116,230) or require an operator to precisely align upstream and downstream sensors along a particular vessel (e.g., U.S. Pat. Nos. 5,680,868 and 5,873,836).

It is often desirable to be able to assess the blood pressure (systolic, diastolic, and mean arterial) in each blood vessel individually rather than in a limb taken as a whole. Further, it is desirable to employ a method for blood pressure assessment based on fundamental physical principles (e.g., occlusive cuff methods) rather than empirical, statistical associations that require correlation factors. Additionally, it is also advantageous to provide a testing system and method that has low operator dependency (particularly with regard to test probe placement) and obviates the need for the use of acoustic coupling gel.

The present invention provides an alternative system and method for vascular testing that overcomes limitations found in the prior art.

### SUMMARY

In one aspect, a vascular testing system includes a first pressure cuff assembly positionable about a limb of a patient and a controller. The first pressure cuff assembly includes a pressure bladder and a first array of acoustic sensors, the first array including a plurality of acoustic sensor elements arranged circumferentially relative to the pressure bladder. The controller is configured to concurrently sense vascular data at two or more discrete testing locations utilizing at least two different sets of the acoustic sensors of the first array each positioned at or near one of the discrete testing locations. Each set of the acoustic sensors includes one or more of the plurality of acoustic sensor elements.

In another aspect, a method of auscultatory vascular testing includes positioning an array of acoustic sensors about a limb of a patient, concurrently sensing data with the array of acoustic sensors, and selecting sensed data outputs of at least two circumferentially spaced sensors of the array as representative of respective physiological conditions of a first blood vessel and a second blood vessel of the limb.

### BRIEF DESCRIPTION OF THE DRAWINGS

FIG. 1 is a schematic block diagram of an embodiment of a vascular testing system according to the present invention.

FIG. 2 is an elevation view of an embodiment of a cuff assembly of the vascular testing system positioned on a limb.

FIGS. 3A-3E are illustrations of different embodiments of the cuff assembly, and associated sensor subassemblies.

FIGS. 4A-4E are illustrations of various embodiments of the cuff assemblies of

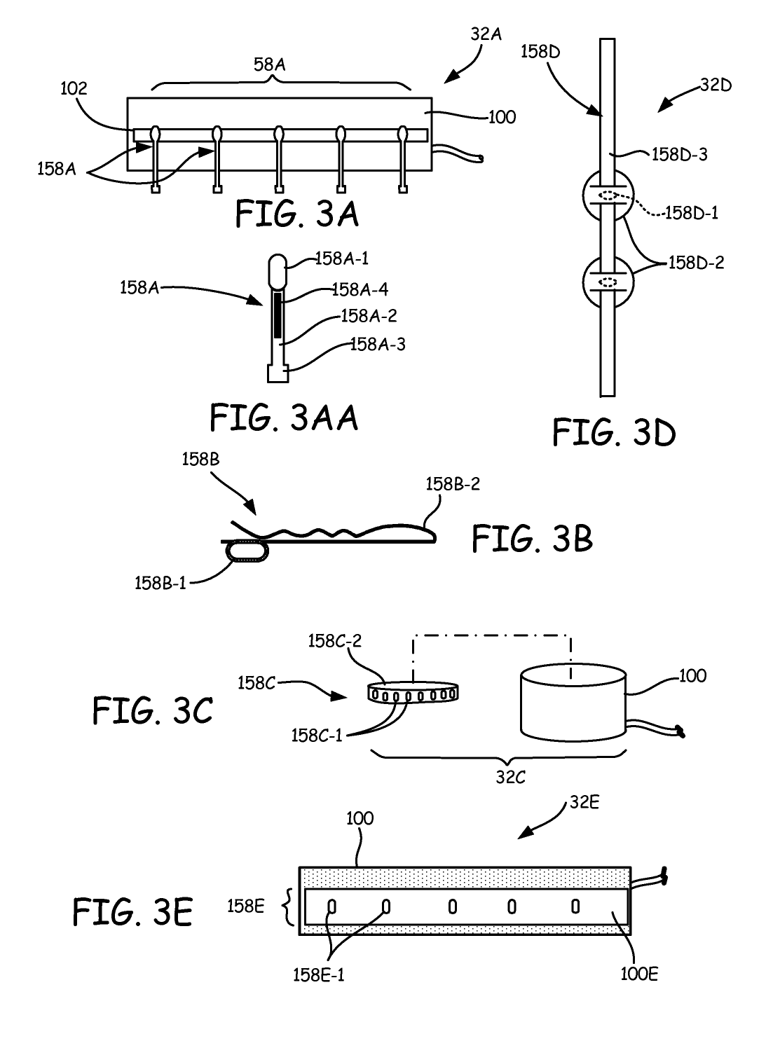

FIGS. 3A-3E positioned on the limb L.

FIGS. 5-5E illustrate additional embodiments of cuff assemblies.

FIG. 6 is a flow chart of one embodiment of a method of performing vascular testing according to the present invention.

FIG. 7A is an example time domain graph of amplitude versus time of an acoustic signal, and FIG. 7B is an example frequency domain graph of amplitude versus frequency for the acoustic signal of FIG. 7A converted to the frequency domain.

FIG. 8 is a flow chart illustrating an embodiment of a method for associating sensors with blood vessels according to the present invention.

FIG. 9 is an example graph of acoustic signal strength, plotted as amplitude versus sensor number.

FIG. 10 is a flow chart illustrating an embodiment of a method of sensor registration.

FIG. 11 is a flow chart that illustrated one embodiment of a method for implementing a cuff application wizard according to the present invention.

FIGS. 12A and 12B are schematic cross-sectional representations of a patient's limb with an embodiment of a cuff assembly applied about the limb.

FIG. 13A is a graph of an example oscillometric cuff pressure signal over time.

FIG. 13B is a graph of a normalized oscillogram of peak-to-trough amplitude versus cuff pressure based on the cuff pressure signal of FIG. 13A.

While the above-identified figures set forth embodiments of the present disclosure, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of the principles of the invention. The figures may not be drawn to scale, and applications and embodiments of the present invention may include features and components not specifically shown in the drawings.

### DETAILED DESCRIPTION

In general, the present invention provides a system and method for performing vessel-specific, noninvasive vascular testing, without requiring precise positioning of test sensors or extensive operator skill. The system and method allows sensing and measurement of blood pressure values (systolic, diastolic, and mean arterial) in individual blood vessels, rather than in a limb taken as a whole. Further, the system and method permits blood pressure assessment based on fundamental physical principles (e.g., occlusive cuff methods) rather than empirical, statistical associations leading to correlation factors. Additionally, the system and method can provide relatively low operator dependency and obviates the need for the use of acoustic coupling gel associated with ultrasonic modalities. In some embodiments, the invention can involve auscultatory testing utilizing multiple acoustic sensors, which can be arranged in one or more generally arrays in circumferential or other patterns. Contralateral measurements can be taken simultaneously or concurrently on different limbs using multiple cuff/sensor assemblies, and a given cuff/sensor assembly can assess multiple blood vessels on a given limb, such as testing both the posterior tibial and anterior tibial arteries in conjunction with contralateral testing. Multiple vessels can be assessed simultaneously to produce bilateral, simultaneous contralateral, simultaneous blood vessel segmental pressure measurements without a need for a Doppler probe. Further, brachial pressures can be assessed with this technique simultaneously leading to near instantaneous pressures and ankle-brachial indexes (ABIs), which can be useful for assessing peripheral arterial disease (PAD) and/or other physiological conditions. Numerous cuff assembly configurations are possible, as discussed below. A method for assisting in sensor and/or pressure cuff placement can further be provided in some embodiment, which can help correct for improper cuff/sensor assembly application or can let a user know that a cuff assembly (including associated sensors) has been misapplied.

Additionally, data fusion approaches are provided that allow different testing modalities to be utilized synergistically. Further, approaches for oscillometric data validation and testing compensation or adjustment are provided.

Various other features and benefits of the present invention will be appreciated in view of the description that follows and the accompanying figures.

Possible Embodiments of System Components

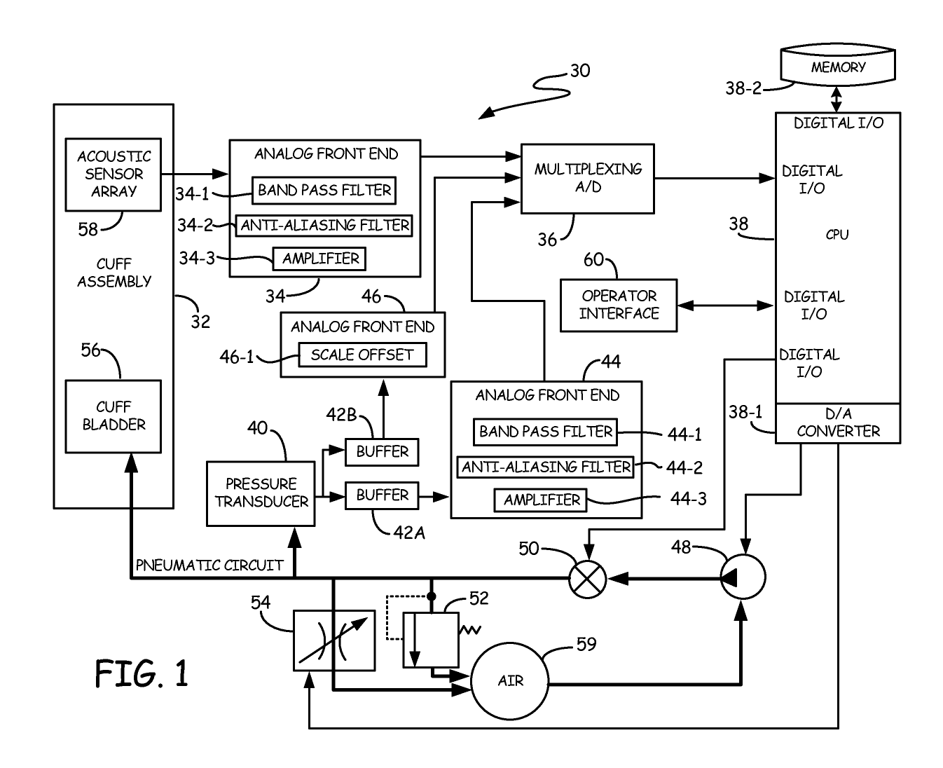

FIG. 1 is a schematic block diagram illustrating an embodiment of a vascular testing system 30. As shown in the embodiment of FIG. 1, the system 30 includes a cuff assembly 32, analog front end circuitry 34, a multiplexing analog to digital (A/D) converter 36, a central processing unit (CPU) 38, a pressure transducer 40, buffers 42A and 42B, analog front end circuitry 44 and analog front end circuitry 46. The system 30 as illustrated further includes a pump 48, a check valve 50, a safety overflow valve 52, and a deflate valve 54. It should be noted that the system 30 shown in FIG. 1 is illustrated merely by way of example and not limitation. For instance, numerous additional components not specifically shown can be included in further embodiments. For instance, additional acoustic, electrocardiogram (EKG), photoplethysmograph (PPG), air pneumoplethysmograph, or other sensors can be provided, and/or signals indicative of acoustic, EKG, PPG, air pneumoplethysmograph or other signals can be received and utilized by the system 30. Moreover, different component configurations can be utilized as desired in further embodiments.

The cuff assembly 32 can include a cuff bladder 56 and at least one acoustic sensor array 58. The cuff assembly 32 can be configured for attachment to a limb of a patient, such as an arm or leg. The cuff bladder 56 can be a conventional pneumatic blood pressure bladder. The cuff bladder 56 can be selectively inflated and deflated using a fluidic (e.g., pneumatic) circuit. The acoustic sensor array 58 can be operably connected to the analog front end circuitry 34, which can include a band pass filter 34-1, an anti-aliasing filter 34-2 and/or an amplifier 34-3, and any other desired circuitry, which in turn is operably connected to the multiplexing A/D converter 36. The acoustic sensor array 58 includes a plurality of sensors that can be placed within, under or upon a body structure of the cuff assembly 32 at or near the location of the cuff bladder 56, as explained further below. In alternatively embodiments, the sensors can be on a separate structure from the cuff bladder 56, and/or can be placed distal (i.e., downstream) from the cuff bladder 56. Any desired number of sensors can be provided in the array 58, such as two to ten or more. Moreover, the acoustic sensor array 58 can be fixed or movable relative to the cuff bladder 56, as explained further below. The acoustic sensors of the array 58 can be microphones or another suitable type of acoustic sensors. In some embodiments, multiple cuff assemblies (or the same or similar configuration as cuff assembly 32) can also be utilized, in order to facilitate simultaneous measurements at multiple locations, such as on contralateral limbs.

Components of the fluidic (e.g., pneumatic) circuit help control selective pressurization of the cuff bladder 56. The pump 48, which can be any suitable type of pneumatic pump (e.g., a voltage controlled fixed displacement pump), can pump fluid (e.g., ambient air 59) through the check valve 50 to the cuff bladder 56. The pump 48 can alternatively be controlled using pulse width modulation (PWM) control using a digital I/O of the CPU 38 (i.e., omitting the D/A converter 38-1). The check valve 50 can be a solenoid valve controllable by the CPU 38 in concert with control of the pump 48, to close the valve 50 when the pump 48 is inactive. Alternatively, the check valve 50 can be of a different type, such as a conventional ball and spring passive check valve. The deflate valve 54, which can be proportionally controlled solenoid valve, can control deflation of the cuff bladder 56 to selectively release fluid (e.g., air 59) from the cuff bladder 56 and the fluidic circuit. Furthermore, the safety overflow valve 52 can allow a maximum pressure threshold to be established, to release fluid (e.g., air 59) to help prevent over-inflation of the cuff bladder 56. Additional components not specifically discussed, such as suitable fluid conduits, tubes, etc. can also be provided with the fluidic circuit. For instance, one or more additional fluidic circuits can be provided for use with additional cuffs and/or different styles of cuffs (e.g., purely oscillometric cuffs).

The pressure transducer 40 can be operatively connected to the fluidic circuit of the system 30, in order to measure pressure at the cuff bladder 56. The pressure transducer 40 can provide one or more output signals to the buffers 42A and 42B. The buffer 42A can provide an output signal to the analog front end circuitry 44, which can include a band pass filter 44-1, an anti-aliasing filter 44-2 and/or an amplifier 44-3, and any other desired circuitry, and which is in turn operatively connected to the multiplexing A/D converter 36. The buffer 42B can provide an output signal to the analog front end circuitry 46, which can include scale offset circuitry 46-1, and any other desired circuitry, and which is in turn operatively connected to the multiplexing A/D converter 36.

The CPU 38 can include one or more discrete processing units each having any number of processing cores, and can function together with suitable memory 38-1 and suitable software or firmware to act as a control module for the vascular testing system 30. The CPU 38 can include an integrated digital to analog (D/A) converter 38-1, which in alternative embodiments can be implemented as stand-alone circuitry separate from the CPU 38. An output through the D/A converter 38-1 can be used to selectively control operation of the pump 48 and the deflate valve 54. Digital input/output (I/O) interfaces can be provided to accept input signals from the multiplexing A/D converter 36 and generate outputs to the check valve 50. An operator interface 60 can be provided that can interface with the CPU 38, in order to provide outputs to an operator using a visual display, audio speaker and/or other output device, as well as to allow operator input to control operation of the system 30. Furthermore, the CPU 38 can be operably connected to suitable communication circuitry to communicate over a wired or wireless connection with other equipment, such as via a network interface, the Internet, etc.

In operation, the vascular testing system 30 can be used to position the cuff assembly 32 in a general location (e.g., in the medial and anterior portion of the patient's ankle) of a desired vessel under test (VUT) without the need to precisely place a particular sensor of the acoustic sensor array 58 directly over the VUT and without the need for acoustic coupling gel (as used with Doppler-based systems). The acoustic sensor array 58 permits multiple blood vessels of a given limb to be sensed simultaneously. Because blood pressure can change from moment to moment, the ability to simultaneously measure the blood pressure in multiple individual vessels (and/or in contralateral limbs) is a significant diagnostic advantage. One reason for this is that one artery in a limb may be occluded (i.e., blocked) and another artery in that same limb may be patent (i.e., open). The occluded artery will have a lower pressure distal to the occlusion than the patent artery. The overall limb pressure will usually reflect the higher of all the artery pressures in a given limb segment. Thus, (overall) limb pressure will not reveal whether an individual artery is blocked (i.e., has lower pressure). Physicians may decide not to intervene if at least one artery is open but, then again, they may decide to intervene. However, physicians cannot consider treatment options fully if they do not know that there is an occluded artery. Lower extremity arterial pathology is often associated with diabetes, so this is quite a prevalent disease state.

Another potential advantage would exist for a patient with an amputated foot or an amputated lower extremity segment. For those devices that use a phototransducer on the toe (i.e., photoplethysmography), the phototransducer system would not be able to measure pressure in addition to its other disadvantage of not being vessel specific at any time. For a Doppler system, the Doppler probe is typically distal to the cuff. Sometimes, for an amputee, there is not room distal to the cuff to even place a Doppler probe. An ausculatory approach (e.g., with the microphones under the cuff) using the system 30 would generally not have these problems or limtiations.

Additionally, sometimes Doppler-based systems cannot read (or hear) flow in an artery. This could be because the flow velocity is too low to measure, because the ultrasound is not focused properly, or because of other reasons. Similarly, an auscultatory approach might, occasionally, fail to measure a signal. In that case (as with Doppler), the system 30 can report the pressure in those arteries whose acoustic signals it can appreciate. The ability to appreciate a signal for one vessel even if not for all vessels is, itself, useful diagnostic information. The fact that one cannot appreciate a signal from one vessel is also diagnostically significant.

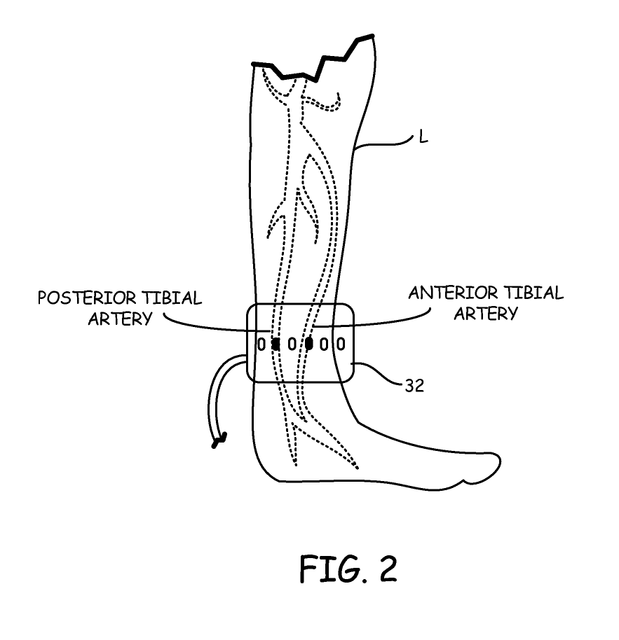

FIG. 2 is an elevation view of the cuff assembly 32 positioned on a limb L, which in the illustrated embodiment is a leg having an anterior tibial artery and a posterior tibial artery. As shown in the illustrated embodiment, the cuff assembly 32 is positioned at an ankle of the limb L. The acoustic sensor array 58 is shown with the sensor closest to the anterior tibial artery and the sensor closest to the posterior tibial artery shown with shading. This way, sets of one or more sensor element can each be positioned at or near discrete testing locations, such as at different blood vessels. In contrast with conventional (e.g., Doppler) means of pressure measurement, a physician or other operator need not possess detailed knowledge of surface anatomy and where to physically locate the cuff assembly 32 to appreciate a signal using the vascular testing system 30. The limb L is illustrated merely by way of example, and the cuff assembly 32 can be positioned one different limbs, such as arms, as well as in different locations along the limb L. Persons of ordinary skill in the art will appreciate that numerous blood vessels are present in a given limb, and that the anterior tibial artery and the posterior tibial artery, which are shown merely by way of example, are found only in lower limbs (i.e., legs). Numerous embodiments of the cuff assembly 32 are possible, as explained further below.

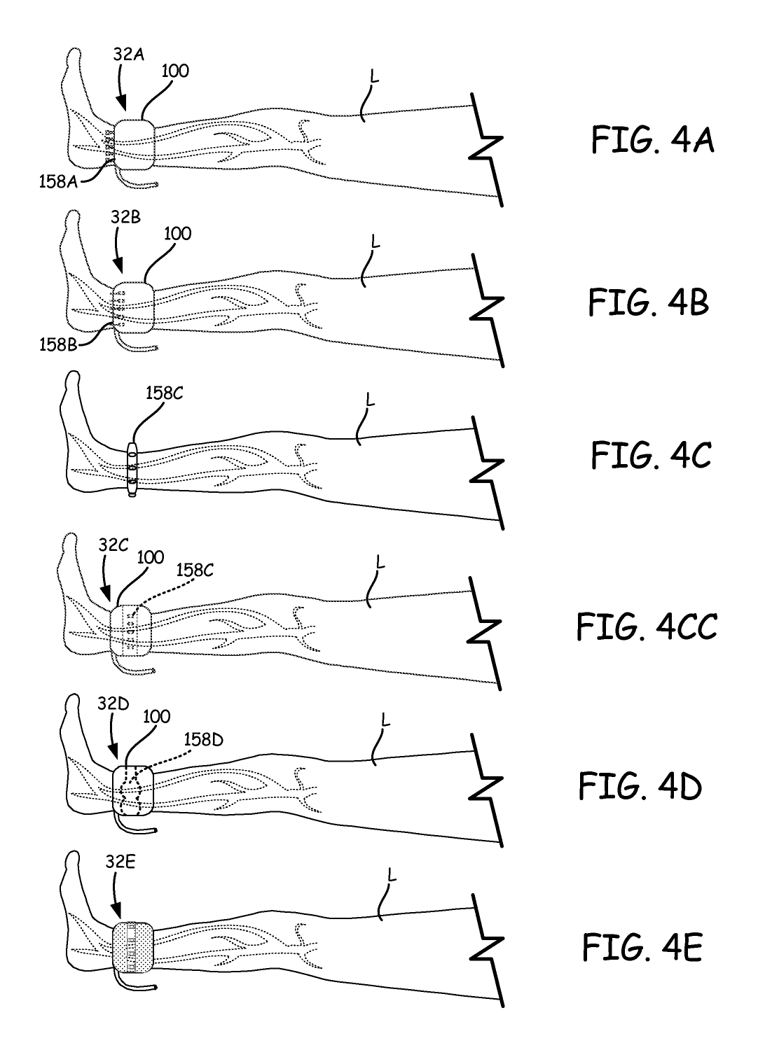

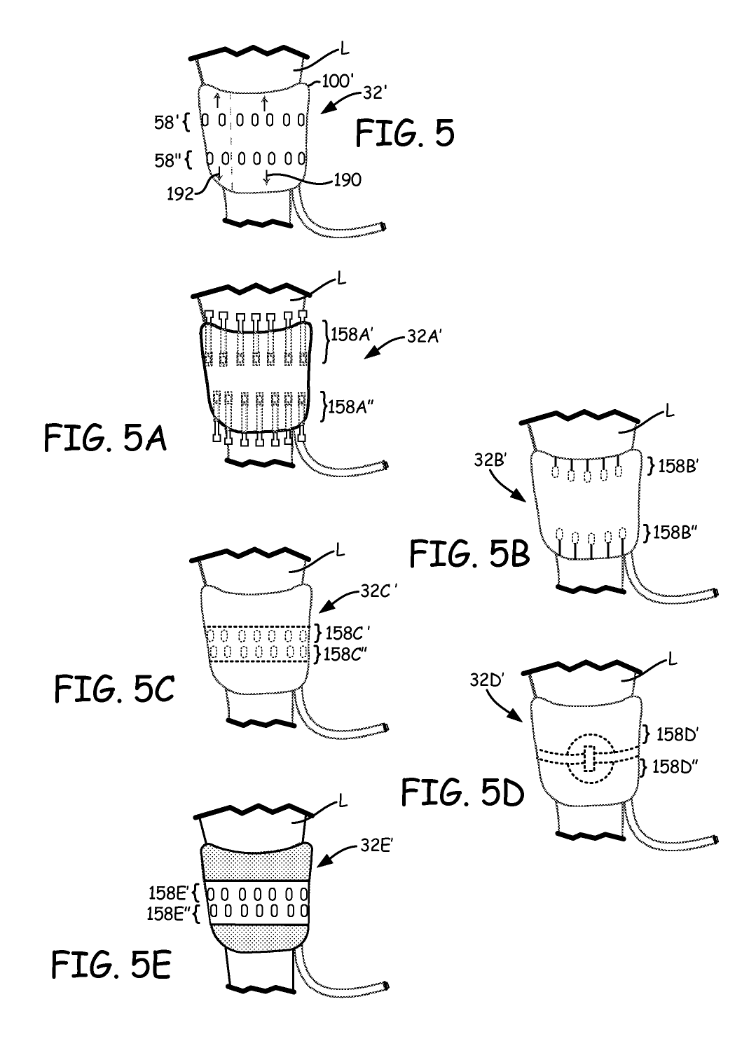

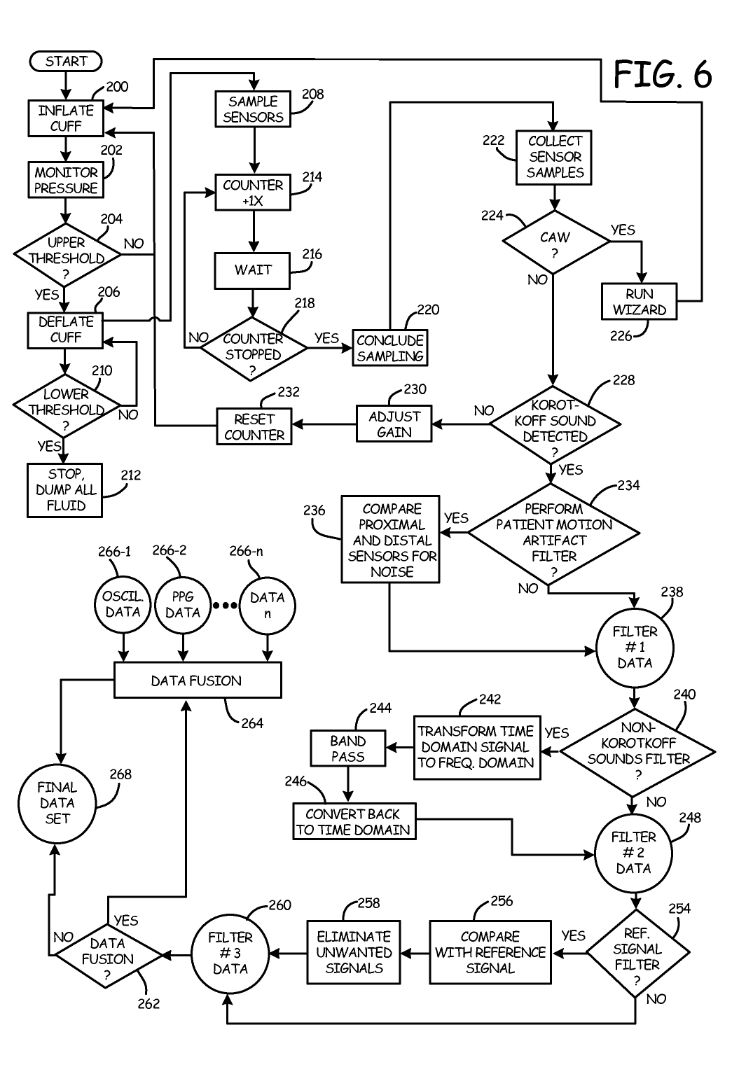

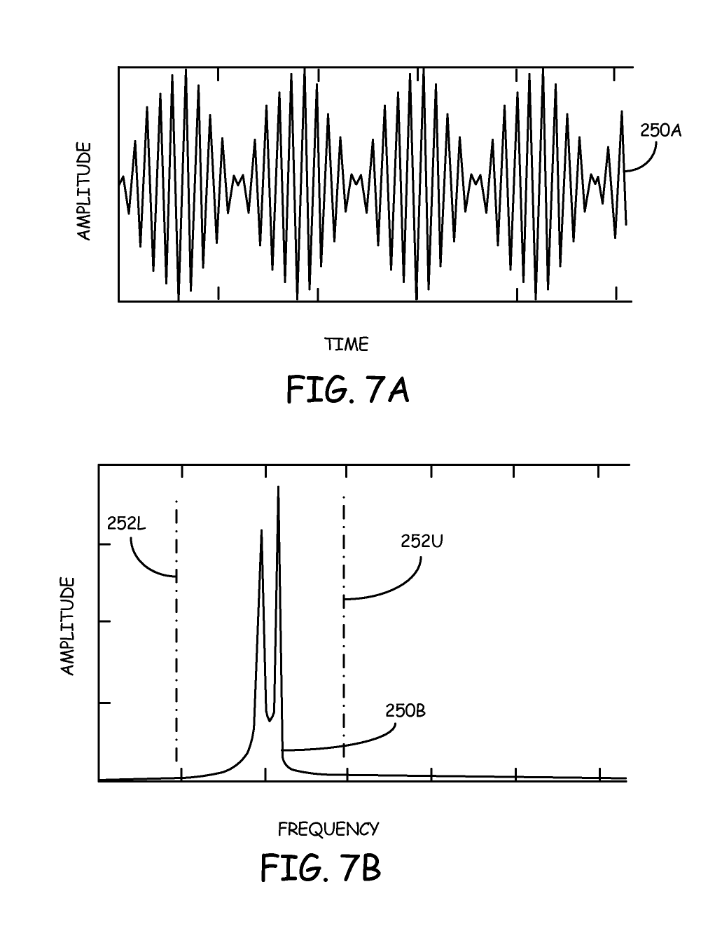

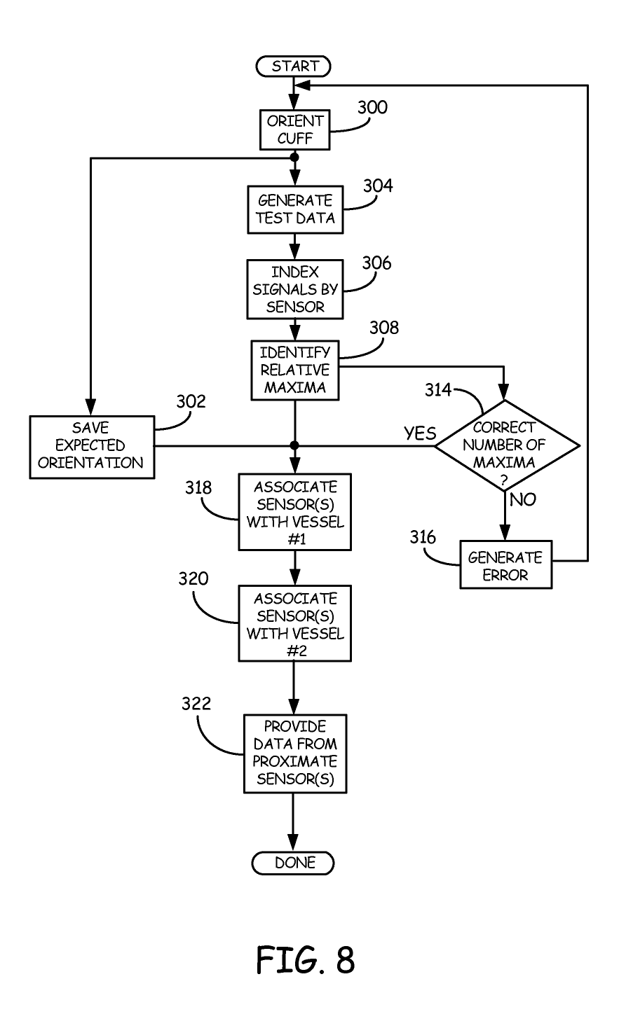

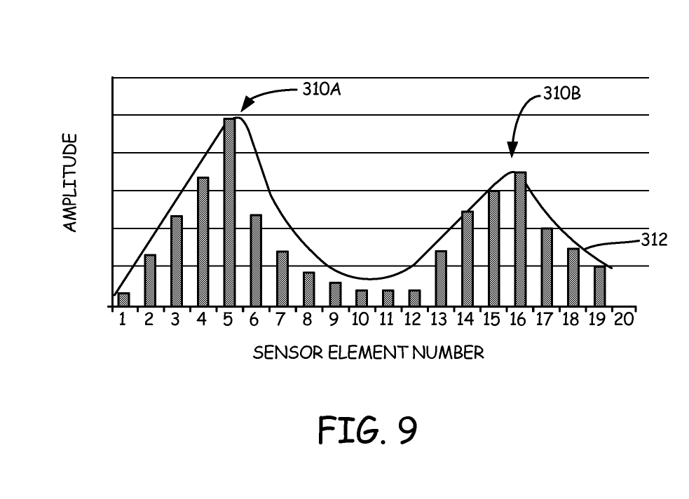

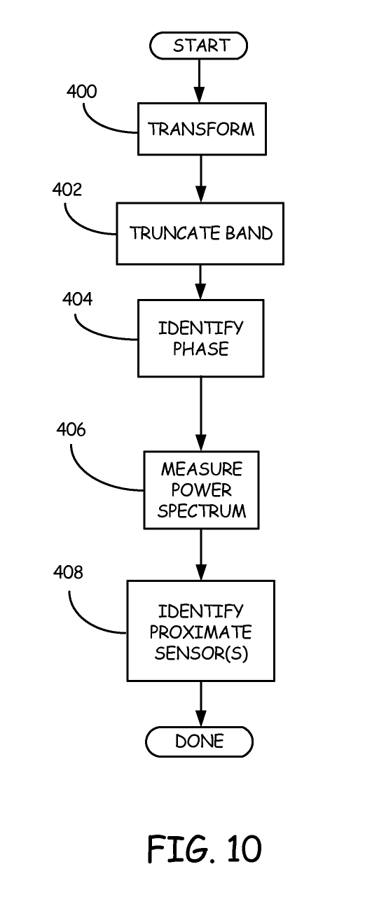

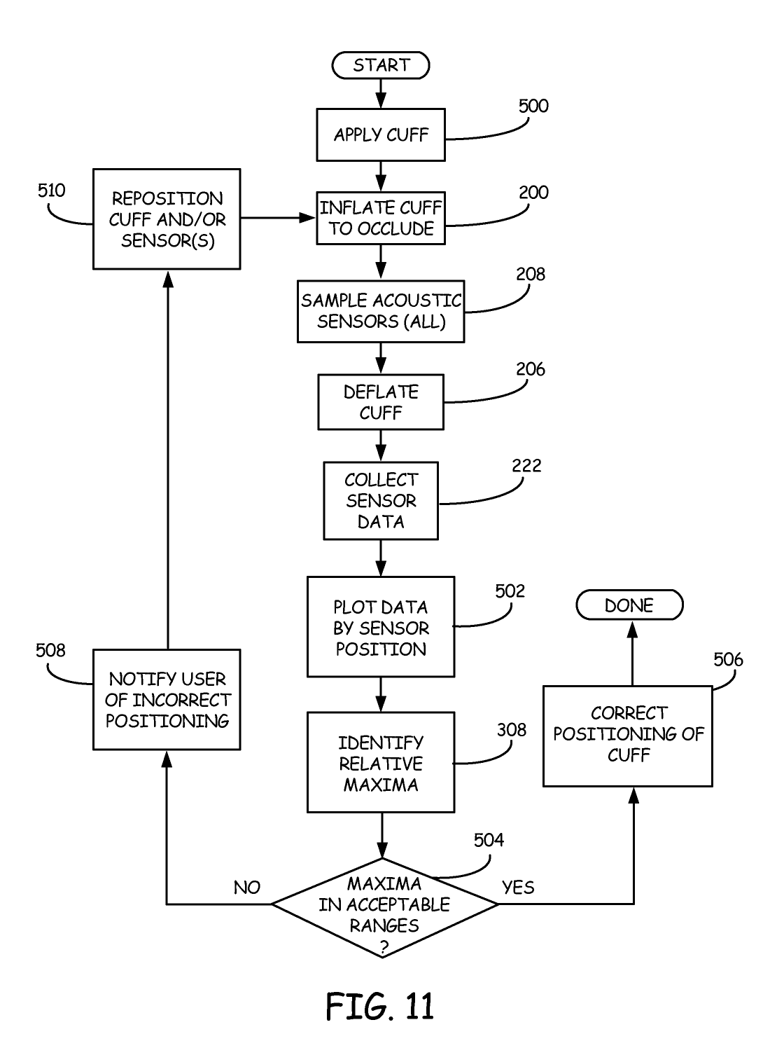

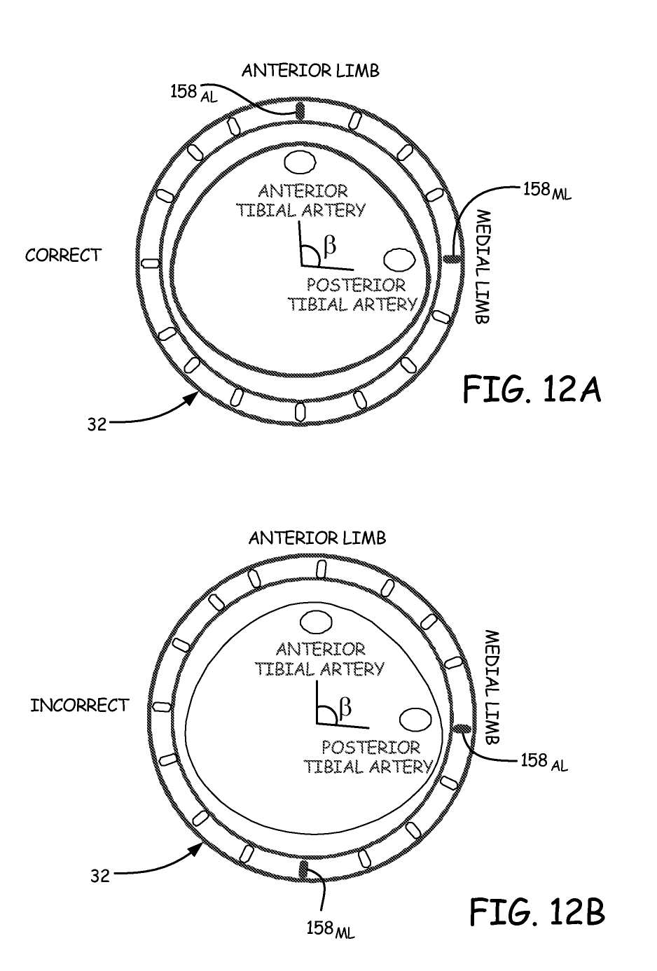

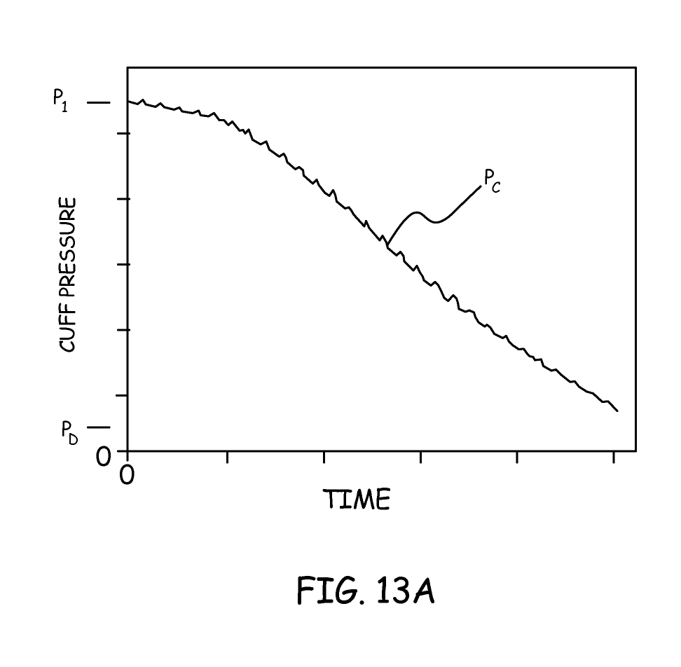

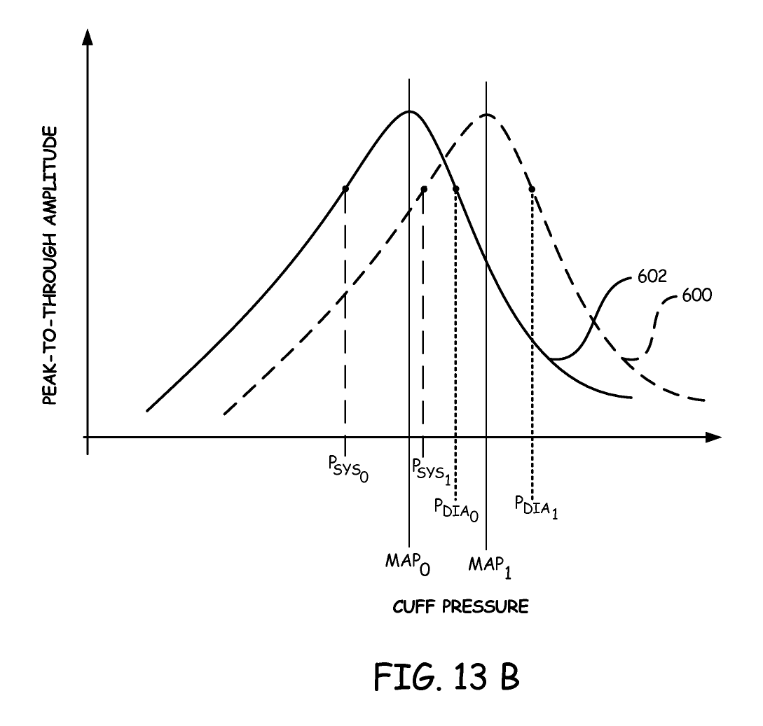

FIGS. 3A-3E are illustrations of different embodiments of cuff assemblies 32A-32E, and associated sensor subassemblies 158A-158E. FIGS. 4A-4E are illustrations of various embodiments of the cuff assemblies 32A-32E of FIGS. 3A-3E positioned on the limb L.

FIG. 3A is a plan view of one embodiment of a cuff assembly 32A having a cuff body 100, an attachment structure 132 and an acoustic sensor array 58, and FIG. 3AA is a plan view of one sensor subassembly 158A of the array 58 (see also FIG. 4A). The cuff body 100 can at least partially cover the cuff bladder 56, which is not visible in FIG. 3A. The array 58A includes a plurality (e.g., five) of sensor subassemblies 158A that can each include a sensor element 158A-1, a body 158A-2, an indicator flag 158A-3 and an attachment structure 158A-4. The indicator flag 158A-3 can be positioned to extend away from the sensor element 158A-1, and the attachment structure 158A-4 can be secured to the body 158A-2. The attachment structure 158A-4 can be a hook-and-loop fastening structure (e.g., Velcro® brand hook-and-loop fastener material), and can engage the corresponding attachment structure 102 on the cuff body 100. In one embodiment, the attachment structure 158A-4 can be a loop part and the attachment structure 102 can be the hook part of the hook-and-loop attachment structures. The attachment structure 102 can extend over a desired area of the cuff body 100, such as to provide a circumferentially-extending sensor positioning area along the cuff body 100 when positioned on the limb L. The attachment structures 102 and 158A-4 allow the sensor subassemblies 158A to be individually repositioned relative to the cuff body 100 (as well as relative to the cuff bladder 56, not shown in FIG. 3A). In that way, any given sensor subassembly 158A can be readily repositioned in a circumferential direction relative to a given blood vessel when the cuff assembly 32A is positioned on the limb L. The indicator flag 158A-3 can protrude from an edge of the cuff body 100, in order for a user to identify locations of the individual sensor subassemblies 158A, as well as to provide a grasping surface. Alternatively, the sensor subassemblies 158A can be positioned such that the sensor elements 158A-1 extend beyond an edge of the body 158A-2, such as to be positioned distal from the body 158A-2 and the cuff bladder 56 (not shown in FIG. 3A). In such distal sensor arrangements, the acoustic sensor is typically still located near the location of the cuff bladder 56. For instance, such a distal acoustic sensor arrangements can, in some embodiments, resemble manual approaches using a stethoscope positioned distal to a pressure cuff or Doppler approaches using a distal Doppler probe.

FIG. 3B is an elevation view of another embodiment of a sensor subassembly 158B, which can be utilized in conjunction with the cuff body 100 of FIG. 3A (the attachment structure 102 of FIG. 3A can be omitted) (see also FIG. 4B). In the illustrated embodiment, the sensor subassembly 158B includes a sensor element 158B-1 mounted to a clip like attachment structure 158B-2, which can resemble a “bobby pin” style hairpin or other suitable style of clip. The clip 158B-2 can be engaged at a desired location on the cuff body 100, typically with a portion extending beyond or about an edge of the cuff body 100, and can be repositioned as desired.

FIG. 3C is an exploded perspective view of another embodiment of a cuff assembly 32C (see also FIGS. 4C and 4CC), which includes the cuff body 100 and a sensor subassembly 158C having a plurality of sensor elements 158C-1 mounted on a sensor band 158C-2. The sensor subassembly 158C can be positioned and adjusted on the limb L separately from the cuff body 100. FIG. 4C shows the sensor subassembly 158C positioned on the limb L, and in FIG. 4CC the cuff body 100 is positioned over the top of the sensor subassembly 158C, that is, with the sensor subassembly 158C located under (i.e., radially inward from) the cuff body 100. In another embodiment, the sensor subassembly 158C can be positioned distally from the cuff body 100, such as being axially spaced along the longitudinal axis of the limb L. The sensor band 158C-2 can be made of any desired material, such as fabric, elastic, hook-and-loop, etc.

FIG. 3D is a plan view of another embodiment of a portion of a cuff assembly 32D that includes a sensor subassembly 158D having a plurality of sensor elements 158D-1 located on acoustic sensor pads 158D-2 and a strap 158D-3. The sensor pads 158D-2 each include at least one acoustic sensor element 158D-1. Any desired number of sensor pads 158D-2 can be provided. The pads 158D-2 can slidingly engage the strap 158D-3, allowing each of the pads 158D-2 to be independently repositioned along the strap 158D-3. The strap 158D-3 and the pads 158D-2 can be used in conjunction with a cuff body 100 (not shown in FIG. 3D), with the cuff body 100 positioned over the top of the other components as shown in FIG. 4D, that is, with the sensor subassembly 158D located under (i.e., radially inward from) the cuff body 100. The sensor subassembly 158D can be positioned and adjusted prior to positioning the cuff body 100. In another embodiment, the sensor subassembly 158D can be positioned distally from the cuff body 100, such as being axially spaced along the longitudinal axis of the limb L.

FIG. 3E is a plan view of yet another embodiment of a cuff assembly 32E that includes a cuff body 100 with an at least partially transparent or translucent window 100E and a sensor subassembly 158 having a plurality of acoustic sensor elements 150E-1. The acoustic sensor elements 150E-1 can be positioned on an underside or within the cuff body 100 and at least partially visible within the window 100E, such that a user can see the locations of the sensor elements 150E-1 through the window 100E as shown in FIG. 4E. In this embodiment, at least a portion of the cuff bladder 56 can be transparent or translucent to facilitate viewing of the sensor elements 150E-1 through the window 100E.

FIGS. 5-5E illustrate additional embodiments of cuff assemblies 32′-32E′ positioned on the limb L. As shown in FIG. 5, the cuff assembly 32′ includes a first acoustic sensor array 58′ and a second acoustic sensor array 58″, which are generally spaced along a longitudinal axis of the limb L. Individual sensors of the arrays 58′ and 58″ are generally circumferentially aligned, to allow upstream and downstream measurements along a given blood vessel. The arrays 58′ and 58″ can be fixed to a cuff body 100′, which also can provide fixed circumferential spacing between individual sensor elements of each array 58′ and 58″. First markings 190 and second markings 192 can be provided on an exterior and/or interior of the cuff body 100′. The markings 190 and 192 can provide an external indication to a user of sensor positioning, thereby allowing the user to rotate the cuff assembly 32′ to a desired circumferential alignment relative to the limb L even if the sensors are not readily visible. For example, the markings 190 can be arrows printed on an exterior of the cuff assembly 32′ to indicate registration of a corresponding sensor or sensors for the anterior tibial artery, and the markings 192 can be can register the corresponding microphone or microphones to the posterior tibial artery. As another example, in addition or in the alternative, the markings 192 can identify a nominal medial area and the markings 190 identify a nominal anterior area. In addition or in the alternative, the markings 190 and/or 192 can include text labels such as “anterior” and “posterior”, etc. The cuff assemblies 32A′-32E′ shown in FIGS. 5A-5E are generally similar to the cuff assemblies 32A-32E described above with respect to FIGS. 3A-3E and 4A-4E, but with first and second acoustic sensor arrays 158A′-158E′ and 158A″-158E″ spaced along the longitudinal axis of the limb L instead of only a single array.

In view of the foregoing, it can be seen that the system 30 allows a plurality of acoustic sensors in an array 58 (having any desired configuration, such as that described above with respect to 58′, 58″, 158A, 158A′, etc.) to be positioned at different circumferential positions about the limb L to simultaneously sense parameters associated with different blood vessels. In some embodiments, with a sufficiently large number of acoustic sensors in the array 58, it can be assumed that at least one sensor of the array 58 will always be positioned directly at a desired vessel test location regardless of orientation of the cuff assembly 32, and the system 30 can analyze data from the sensors array 58 to automatically determine which sensors are located at the desired vessels under test. Alternatively, individual sensors or the entire array 58 can be repositionable so that an operator can position sensors in the array 58 at locations corresponding to desired individual blood vessels. The exact position of vessels desired to be tested in the limb L will vary depending on the type of limb (e.g., leg or arm) and patient physiology (e.g., size of limb). For instance, circumferential sensor spacing for a patient having a relatively large diameter (and circumference) for the limb L can be different from that of another patient having a relatively small diameter (and circumference) for the limb L, in order to accommodate associated differences in blood vessel positions. Operation of the system 30 is discussed further below.

Testing Protocol

FIG. 6 is a flow chart illustrating one embodiment of a method of performing a vascular testing, which can utilize the system 30. Operation of the system 30 can be governed at least partially through the CPU 38. It should be noted that the illustrated embodiment of the method is shown merely by way of example and not limitation. Persons of ordinary skill in the art will recognize that various illustrated steps can be performed concurrently, or in a different order than shown. Moreover, additional steps not specifically listed can also be performed as desired for particular applications, and illustrated steps can be omitted in some embodiments. For instance, testing according to the present invention could be implemented in conjunction with the systems and methods disclosed in commonly-assigned U.S. Pat. Nos. 7,983,930 and 8,229,762, to provide remote diagnosis and other additional features.

Initially, one or more cuff assemblies 32 (any embodiment of a cuff assembly and its subcomponents can be utilized, although citations are generally only made to reference numbers of one embodiment, for simplicity) are positioned at desired testing locations on the patient, such as at the limb L. Cuff markings can be utilized to help improve accuracy of cuff and sensor orientation, such as to position an “anterior” label of the cuff assembly 32 at a most anterior region of the limb L. In the description that follows, reference is made to steps taken for a given cuff assembly 32, though it should be recognized that the same or similar steps can be taken for each cuff assembly 32 when multiple cuff assemblies 32 are utilized for simultaneous testing at multiple test locations on the patient.

After the cuff assembly 32 has been positioned (for each limb L under test), the cuff bladder 56 can be inflated using the pump 48 (step 200). Cuff pressure can be monitored using the pressure transducer 40 (step 202). The cuff bladder 56 can be inflated to a super-systolic (relative to brachial pressure) level or a fixed pre-set level, which can be assessed against an upper pressure threshold (step 204). After the upper threshold is reached, the cuff bladder 56 can be deflated using the deflate valve 54 (step 206). In one embodiment, the cuff bladder 56 can be deflated at approximately 2-3 mmHg/sec or approximately 2-3 mmHg/heart beat. At least during the deflation process, all acoustic sensors of the array 58 can be monitored (e.g., polled or sampled) and the pressure transducer 40 can continued to be monitored (e.g., polled or sampled) for cuff inflation pressure (step 208). Signals from the acoustic sensor array 58 can be amplified and filtered using the analog front end circuitry 34, as desired. Likewise, signals from the pressure transducer 40 can be buffered in the buffer 42A and amplified and filtered with the analog front end circuitry 44, as well as buffered in the buffer 44B and offset scaled using the analog front end circuitry 46, as desired. Numerous filter topologies (e.g. Butterworth, Sallen-Key) may be used without departing from the scope of the invention. Further, these filters may be implemented via analog or discrete-time means without departing from the scope of the invention. In embodiments where multiple arrays of sensors are used (e.g., as described and shown with respect to cuff assemblies 32′-32E′), separate signals for each sensor array (e.g., 58′ and 58″) can be separately handled. Sensor data, after any desired front-end processing, can be stored in the memory 38-1, for instance, in individual time-indexed arrays for each limb and sensor array.

During deflation, cuff pressure is monitored for a lower threshold (e.g. when cuff air inflation pressure reaches approximately 10 mmHg) (step 210). When the lower threshold is reached, marking an end of the deflation process, all fluid can be dumped (i.e., removed) from the system 30 (step 212), causing all blood pressure cuffs 32 to be completed deflated.

As the cuff bladder 56 is deflated and the acoustic sensor array(s) 58 are sampled, a counter can be run. For instance, a counter can increment by 1× intervals (e.g., 100 ms) (step 214), waiting for the 1× interval (step 216) and then incrementing the counter again if the counter has not stopped (step 218). The counter can be cued to stop when the cuff bladder deflation process has concluded at step 212, or alternatively at another time (e.g., when the counter reaches a given threshold). When the counter is stopped, sampling of the acoustic sensor array(s) 58 can be concluded (step 220). At that point, a set of acoustic sensor samples have been collected and stored that can then be analyzed (step 222).

A determination can be made as to whether a cuff application wizard (CAW) protocol should be performed (step 224). If desired, the CAW protocol can be run (step 226), with the process returning to step 200 to re-inflate the cuff bladder 56 and re-sample the acoustic sensor array(s) 58 after the CAW protocol is complete. The CAW protocol is explained further elsewhere in the present application.

If the CAW protocol is not desired (or has previously been completed), the process can proceed to a determination as whether a Korotkoff sound is detected (step 228), which can involve a determination as to whether a sensor in an acoustic sensor array 58, or a circumferentially-aligned proximal/distal acoustic sensor pair of arrays 58′ and 58″, produce a signal or signals that meets one or more desired characteristics, the system can register the sensor data as including a legitimate Korotkoff sound. If a signal does not meet the desired characteristics, gain can be adjusted (step 230), the counter reset (step 323) and the process can return to step 200 to repeat testing. In this way, automatic gain control can be provided, and genuine Korotkoff sounds can be better differentiated from interfering signals. The desired signal characteristics can be one or more parameters, such as signal amplitude, signal frequency, or signal morphology (e.g., waveform shape when the signal is plotted). For amplitude and/or frequency parameters, for instance, a desired threshold can be established. Failure to appreciate a sound signal meeting one or more of the desired signal characteristics can cause signal amplification to occur to increase sensitivity when a testing procedure is repeated. Amplification beyond a specified maximum can be prohibited.

If a legitimate Korotkoff sound has been detected, the system 30 can perform any number of desired filtering procedures on the available data. The following are examples of possible filtering protocols, though in alternative embodiments few or greater numbers of filtering protocols can be used, and the filtering protocols can be performed in nearly any desired order, and/or concurrently.

If the system 30 is configured with proximal and distal sensor subassemblies in the cuff assembly (as with cuff assemblies 32′-32E′), then a patient motion artifact filter can be performed as desired (step 234). The patient motion artifact filter can involve comparing data for two or more proximal/distal sensors (e.g., of two or more spaced sensor arrays, such as the acoustic sensor arrays 58′ and 58″) that are substantially circumferentially aligned relative to the limb L (step 236), so as to be all substantially aligned with a given vessel under test. During step 236, time correlation (i.e. time difference) between one or more sets of circumferentially-aligned proximal/distal sensors can be compared to determine any phase difference between each proximal/distal sensor pair, which can be accomplished in multiple ways without departing from the scope of the invention. Illustratively, a correlation function can be applied to assess the time/phase difference, transit time directly can be assessed directly, or the two signals can be multiplied together. After any desired time correlation is performed, all acoustic signals that occur simultaneously in each proximal/distal sensor pair can be ignored or discarded, because such signals are reflective of common mode noise (e.g. patient motion artifact) rather than blood flow-related signals. Thus, if all proximal and distal sensors register an acoustic signal simultaneously, that signal can be identified unwanted noise and can be rejected as not being a genuine Korotkoff sound. If all sensors register a signal with a time delay indicative of blood pulse velocity between the proximal and distal locations, this would indicate a genuine Korotkoff sound suitable for diagnostic or other testing purposes. In this way, noise can be reduced and testing accuracy improved. After any desired filtering at steps 234 and 236 are performed, filter data is output (step 238).

A decision can be made to determine whether to filter an acoustic signal for non-Korotkoff sounds (step 240). Such a process can help improve rejection of signals that are not Korotkoff sounds. If performed, one embodiment can employ a time domain filter (e.g. analog active or passive bandpass or low pass electronic filter, switched capacitor filter, and/or discrete time domain software filter). In another embodiment, the non-Korotkoff sounds filter can include performing a transform of a time domain signal to a frequency domain (step 242), deleting signals outside of an expected or characteristic range for Korotkoff sounds (step 244), and then converting the filtered frequency domain signal back to the time domain (step 246). After any desired filtering at steps 240-246 are performed, filter data is output (step 248). The transform can be a short time fast Fourier transform (FFT) (e.g. Welch Transform) of time-segmented arrays of time domain data or can be a Fourier Transform of the entire time domain dataset generated during cuff deflation, for example. FIG. 7A is an example time domain graph of amplitude versus time of an acoustic signal 250A, and FIG. 7B is an example frequency domain graph of amplitude versus frequency for an acoustic signal 250B, which represents the acoustic signal 250A converted to the frequency domain. The band pass filtering can be performed relative to a lower bound 252L and an upper bound 252U, which can each be selected as desired to provide suitable filtering for a given application. Typically, suppression of undesired signals is accomplished through the use of electronic or discrete time filters. While such known filters can improve the signal-to-noise ratio and can be used in conjunction with the present invention, such filters will not eliminate out-of-band signals and will not attenuate, at all, unwanted in-band noise.

Returning to the example flow chart of FIG. 6, another possible filter can be performed using at least one reference signal (step 254). This step can help reduce unwanted noise. One or more reference signals can be provided that are compared for frequency content, timing, and/or amplitude, with a given acoustic sensor signal for validation as a genuine Korotkoff sound (step 256). Illustratively, a given reference signal can be provided by employing an acoustic sensor (e.g., microphone) placed on the patient's brachial artery, an EKG signal, photoplethysmograph (PPG) signal, an air pneumoplethysmograph signal, etc. As a result of the comparison, unwanted signals (i.e., unwanted portions of a given sensor signal) can be eliminated or zeroed out (step 258). After any desired filtering at steps 254, 256 and 258 are performed, filter data is output (step 260).

Data generated using auscultatory methods (e.g., filtered data from step 260) can optionally be combined with data obtained through other modalities as part of a data fusion process. A determination can be made as to whether data fusion is desired (step 262). If desired, data fusion can be performed (step 264). The data fusion process can include accepting data inputs (steps 266-1 to 266-n) from one or more modalities, including oscillometric data (266-1), PPG (photoplethysmographic) data (266-2), tonometric data (not shown), etc., up to an nth data input (266-n). For instance, using traditional oscillometric methods (which could be performed using the system 30 and the cuff assembly 32), a systolic arterial blood pressure for the limb L can be measured for reference compared to the heretofore described auscultatory method.

Data fusion according to the present invention can provide for various comparisons and data validation subprocesses. For example, if an auscultatory test and analysis algorithm is unable to produce an arterial blood pressure value, an oscillometric arterial blood pressure can be displayed (e.g., using operator interface 60) for review. Moreover, such data fusion can help reduce patient motion and other artifacts in test data as well as to help ensure suitable sensitivity under low blood flow (e.g., stenosis, hypovolemic shock) conditions, which can be challenging for both oscillometric and Doppler-based techniques. A switching protocol can be provided for highlighting a given sensing modality under given conditions. In addition or in the alternative, an oscillometric pulse wave signal can be reviewed and compared in relation to auscultatory data to ensure that morphology, size, and frequency content are appropriate for human physiology. An indication can then be provided as a quality-of-signal measure for blood pressure accuracy. One example of oscillometric waveform morphology analysis is described further below. In addition or in the alternative, a voting process can be conducted among some or all received data inputs from different modalities. A weighting or voting approach can be employed to select a best possible candidate from available data, based on signal qualities, suitability of particular sensing modalities under given physiological conditions, etc.

Oscillometric techniques can involve, at least in part, an assessment of a pressure signal as an occlusive cuff (e.g., cuff assembly 32 or another cuff assembly) deflates. If the assessment suggests that no readable pulse signal is present, the system 30 can produce a message to the user (e.g., using operator interface 60) indicating that no signal could be appreciated. Similarly, the Korotkoff/auscultatory approach can involve an assessment of sound signal(s) to ensure that the signal(s) is/are a genuine Korotkoff sound, as described above. Failure to ultimately appreciate a genuine Korotkoff sound can be indicated to the user (e.g., using operator interface 60) that no readable pulse is present. Because the criteria for Korotkoff and oscillometric pulse signals are different from one another, there are instances for which oscillometry may perceive a signal but microphone methods will not and there are cases for which microphone methods will receive a signal but oscillometric ones will not. There are multiple ways in which data fusion can be accomplished to produce a result superior to either method separately. In one embodiment, the oscillometric signal can be correlated in time with an auscultatory approach. Thus, the oscillometric signal can help validate and confirm the presence of an auscultatory signal. Further discussion of oscillometric methods is provided below.

Numerous other processes can be performed using data the system 30. For example, use of pulse wave velocity (PWV) calculations can optionally be used to screen patients for physiological conditions that may affect test results. In embodiments of the system 30 that include a cuff assembly like cuff assembly 32′, with proximal and distal sensors, PWV can optionally be calculated using the formula of equation 2:

$$PWV = \frac{{D}_{P - D}}{P} \qquad ( 2 )$$

where D_{P-D} is Proximal-to-Distal Sensor distance, and P is phase difference between the proximal and distal sensors. Alternatively, another type of sensor pair can be used to determine phase difference (i.e., transit time) in order to assess PWV without departing from the scope of the invention, such as through the use of photo-sensors (e.g., PPG sensors) that can be located on the patient's upper thigh and ankle, respectively.

One potential problem with sphygmomanometry relates to the problem of pseudo-hypertension. When arteries become at least partially calcified or otherwise hardened, elevated cuff pressures are needed to completely occlude blood flow in the patient's limb L. All occlusive cuff techniques suffer from this potential source of inaccuracy. Thus, for Doppler and auscultatory pressures (oscillometric pressure approaches are also susceptible to this issue but the effect can be compensated for by modifying the systolic and diastolic fractions based on compliance/stiffness data in an approach described below), measured arterial systolic pressure may be elevated with respect to actual intravascular systolic arterial pressure as measured with a catheter (i.e., invasively). This false elevation can lead to false negative results (i.e., normal pressure in cases of arterial insufficiency) which can lead to a misdiagnosis by medical personnel relying on such test data. As a quality check, arterial stiffness can be assessed by the system 30 through analysis of PWV in the limb L under test. PWV generally refers to the speed with which the blood pressure wave travels distally from the heart. It is often multiples of the blood velocity. For calcified or very low compliance arteries, the PWV will be elevated. The system 30 can measure PWV to assess arterial compliance. If compliance is lower than a given threshold, the system 30 can send a message (e.g., using operator interface 60) that warns the user that measured pressure may be lower than indicated due to calcification (or other sources of arterial stiffness) or due to the presence of a synthetic graft. Alternatively, the system 30 can automatically reject test results that fail to pass the PWV threshold. In this way, clinicians can interpret measured pressures more appropriately with a concomitant reduction in false negatives. This can also lead to improved correlation between Doppler pressures and pressures measured through other technologies. In the prior art, PWV is used as a metric in its own right to assess vascular status. According to the present invention, PWV can in addition or in the alternative be used as a quality check for occlusive blood pressure measurement.

Alternatively or in addition, the PWV can be used to assess blood vessel stiffness. For instance, a compensation formula (empirically derived) can be used to adjust measured arterial blood pressure to consider contributions due to relative stiffness of the arterial wall. If arterial stiffness exceeds a threshold value, compensation may not be possible and the system can generate a signal indicating that use of an occlusive cuff approach for measuring arterial blood pressure is contraindicated.

Alternatively or in addition, data obtained using another testing modality (e.g., oscillometric testing) can be used to help screen test data as a function of vessel stiffness or other physiologic factors. For instance, oscillometric screening techniques described below can be utilized to help screen auscultatory test data.

After test data is collected (e.g., step 268), resulting systolic and diastolic arterial blood pressures can be indicated to the user along with measures of PWV and arterial stiffness, such as by providing an output of information to the operator interface 60 or communicated to other equipment external to the system 30. Further, the recorded brachial arterial systolic blood pressures can be noted so as to calculate the ABI (i.e., ankle systolic pressure/brachial systolic pressure).

As noted above, time-indexed pressure signal data and time indexed acoustic sensor array data can be stored by the system 30 in memory 38-2 (or any other suitable location) for the vessel(s) under test (VUT). As the cuff pressure transducer(s) 40 is/are interrogated, the system 30 notes the decrease in air pressure (correlating with the now-completed cuff deflation process). For each pressure sensor element of the array(s) 58, the associated acoustic sensor signal(s) is/are reviewed to identify the first instance of legitimate Korotkoff sounds. At this instance of legitimate Korotkoff sounds, the pressure in the system (as time-indexed to the associated acoustic sensor(s)) can be reported as being equal to an arterial systolic blood pressure, for the given VUT. Further, when the Korotkoff sounds cease to be observed, the pressure from the pressure transducer 40 (from the associated time-index point(s)) can be reported as being equal to an arterial diastolic blood pressure, for the given VUT. The process for any individual vessel for identifying blood pressure can be conventional, though the system 30 allows for simultaneous blood pressure measurements of multiple vessels in the same limb L and in different (e.g., contralateral) limbs, which goes beyond the capabilities of prior art systems.

Sensor-to-Vessel Associations

One aspect of the present invention can involve sensing sounds (e.g., Korotkoff sounds) from a multiplicity of acoustic sensors in a given acoustic sensor array 58 (or any other sensor array embodiment), which are arranged in a circumferential pattern around a long axis of the limb L under test, such as shown in FIGS. 4A-5E. Generally speaking, the system 30 can sense amplitudes of each sound signal from vessels under test sensed by each sensor element (e.g., 158A-1 to 158E-1) and can then identify/locate points of maximum amplitude in a frequency band of interest. For instance, there will typically be two relative maximum points correlating with sensor elements (e.g., two of the sensor elements 158A-1 to 158E-1) located proximate to the primary blood vessels (i.e., arteries) of interest in the limb L, which, for testing on a patient's ankle, will generally be the anterior tibial and posterior tibial arteries, respectively. The sensor array 58 can be registered to the cuff assembly 32 and its external markings or indicia (e.g., markings 190 and 192, one or more indicator flags 158A-3, etc.). In this way, the sensor element or elements located closest to the reflected surface of primary arteries can be identified, which can help obviate the need for the clinician/user to adjust the device for each patient under test, thereby avoiding the need for precise positioning of the sensors. Various details and benefits of a sensor registration method of the present invention are described further below.

FIG. 8 is a flow chart illustrating an embodiment of a method for associating sensors with blood vessels. It should be noted that the method as illustrated in FIG. 8 is shown merely by way of example and not limitation. Persons of ordinary skill in the art will recognize that various illustrated steps can be performed concurrently, or in a different order than shown. Moreover, additional steps not specifically listed can also be performed as desired for particular applications, and illustrated steps can be omitted in some embodiments.

Initially, the cuff assembly 32 (or any other embodiment of a cuff assembly) can be positioned on a patient's limb L and oriented in a nominal orientation (step 300). The nominal orientation can be guided by the markings 190 and 192 or at least one of the indicator flags 158A-3, for example, in order to provide a rough and approximate orientation (e.g., circumferential orientation) of particular sensor elements in the acoustic sensor array 58 relative to the limb L. In this way, an expected orientation of the cuff assembly 32 relative to the limb L can be saved by the system 30 (step 302). The saved expected orientation can be pre-defined by the system 30, for instance, based upon the particular markings provided on the cuff assembly 32 and a known location of a given sensor element or elements relative to the markings, or can be saved for individual test procedures based on feedback associated with that test. For instance, in one embodiment, a sensor element #1 of the array 58 can be registered in saved information as being associated with a nominal posterior, medial or anterior location, and for that saved information the locations of other sensors in the array 58 relative to the sensor element #1 can be known or estimated by the system 30 (where individual sensors elements can be individually repositioned, registered sensor locations will be subject to greater variations from actual locations).

Test data can be generated (step 304). The test data can be generated using the methods described above with respect to FIG. 6. Acoustic signals can then be indexed by sensor, as well as by time (step 306). In this way data collected during testing can be analyzed individually for each sensor element in a given sensor array 58.

After sensor data has been collected and indexed (as well as after any desired pre-processing, such as anti-aliasing, bandpass filtering, and/or amplification), loci of relative maxima among sensor data for a given sensor array 58 can be identified (step 308). Generally, the sensor(s) with a relative maximum amplitude signal or power spectrum amplitude (e.g., at or near the time of the appearance of Korotkoff sounds or at another desired time index) can provide an indication of the associated sensor(s) that is/are most directly able to sense sound from the location nominal vessel, such as those emanating from the posterior tibial artery or from the anterior tibial artery, thereby providing for vessel specific outcomes. A variety of approaches can be used to identify relative maxima. For instance, data can be plotted or graphed and the resultant plot or graph analyzed to identify relative maxima. Alternatively, analysis of raw data can be performed mathematically to determine relative maxima. Additionally, a power spectrum analysis can be performed, as described further below. Identification of relative maxima can be accomplished by analysis of each and every sensor element individually, or alternatively using an average or median of every n sensors to identify regions (each made up of multiple sensor elements) having relative maximum signal strengths. If two or more adjacent sensors return a signal of equal or substantially equal amplitude, the system 30 can average the results by region, select a median (i.e., middle) sensor element, select all of those sensors with the closest large vessel, etc. Although the phases of the various sensors in a given array should be approximately the same, the system 30 could optionally identify the relative phase of each sensor element signal in the time domain though a correlation function or convolution.

FIG. 9 is an example graph of acoustic signal strength, plotted as amplitude versus sensor number, where each sensor number corresponds to a sensor element of the acoustic sensor array 58 when applied to an ankle location on a patient's leg. The graph in FIG. 9 can represent signals from the array 58 at the appearance of Korotkoff sounds. As shown in FIG. 9, signal amplitude for the various sensor elements varies, and sensor elements #5 and #16 provide relative maxima 310A and 310B. The relative maxima can correspond to sensors positioned most proximate to individual blood vessels, such as the posterior tibial artery and the anterior tibial artery (or other vessels) (see also, e.g., FIG. 12A). Analysis of the graph can allow identification of these relative maxima, which in turn permits differentiation of Korotkoff sounds from different vessels. Optionally, a curve 312 can be fit to the data of the graph to facilitate identification of the relative maxima 310A and 310B. It should be noted that any suitable methodology for identifying relative maxima can be employed, as desired for particular applications.

The method can optionally screen the identified relative maxima (e.g., 310A and 310B), in order to verify whether an anticipated number of loci were identified for the particular testing location (step 314). For example, where testing is performed on an ankle, two relative maxima are expected, corresponding to the posterior tibial artery and the anterior tibial artery. If a greater or lesser number of maxima are identified than expected, an error signal can be generated (step 316) and the testing can optionally be repeated. Where the testing location is an ankle, each of the aforementioned loci of acoustic sensor element signals will be located proximate to either the posterior tibial artery or the anterior tibial artery. Alternatively, step 314 could involve ignoring or eliminating one or more relative maxima instead of returning an error signal.

After the loci of the relative maxima (e.g., 310A and 310B) are identified, particular sensors of a given array 58 can be associated with individual vessels. For instance, one or more sensor elements of the array 58 can be associated with a first vessel (e.g., anterior tibial artery) (step 318) and one or more other sensor elements of the array 58 can be associated with a second vessel (e.g., posterior tibial artery) (step 320). The associations made at steps 318 and 320 can be to a single sensor element for each relative maxima, for instance, with respect to the example shown in FIG. 9, the relative maximum 310A can be associated with sensor element #5 (only) and relative maximum 310B can be associated with sensor element #16 (only). Alternatively, a plurality of sensors (e.g., three) can be associated with any of the relative maxima, as a function of the sensors closest to the loci of relatively highest amplitude. For instance, with respect to the example shown in FIG. 9, the relative maximum 310A can be associated with sensor elements #4-6 and relative maximum 310B can be associated with sensor elements #15-17.

Associations between sensor(s) and vessels can be performed with reference to stored data regarding expected orientation of the cuff assembly 32, as discussed above with respect to step 302. For example, if an “anterior” registration mark (e.g., markings 190) on the cuff assembly 32 is registered to a known sensor number (illustratively, sensor #1, with reference to the sensor numbers shown in FIG. 9), the locus of maximum amplitude closest to that sensor number (e.g., sensor #1) can be associated with an anterior blood vessel (e.g., the anterior tibial artery). One the first vessel is associated with a particular sensor element or sensor elements, the second vessel can be associated with another sensor element or group of sensor elements, which can be based upon a known or expected relationship between blood vessels at a given testing location. In this way, the sensor elements closest to the vessels to be tested (e.g. anterior tibial artery and posterior tibial artery, for testing at a patient's ankle) can be determined by the system 30, and vessel specific blood pressure measurements can be implemented.

FIG. 10 is a flow chart illustrating an embodiment of a method of sensor registration. Generally, in order to identify the point or points of maximum signal strength among signals from a given acoustic sensor array 58, in addition to any desired analog or discrete time filtering to reduce or eliminate non-Korotkoff sounds, the system 30 can analyze phases of signals emanating from each sensor in the array 58. It should be noted that the method as illustrated in FIG. 10 is shown merely by way of example and not limitation. Persons of ordinary skill in the art will recognize that various illustrated steps can be performed concurrently, or in a different order than shown. Moreover, additional steps not specifically listed can also be performed as desired for particular applications, and illustrated steps can be omitted in some embodiments.

A transform can be performed to convert time domain acoustic sensor signals to the frequency domain (step 400). For instance, a fast Fourier transform (FFT) or discrete Fourier transform (DFT) can be performed, or any other desired transform. Then out-of-band (i.e., outside a specified frequency range) signals can be truncated (i.e., eliminated) from each sensor's spectrum for the given array 58 (step 402). This truncation can be in addition to other truncation or band pass filtering performed elsewhere in a testing process. For each sensor signal, the phase can be identified and similar phases identified (step 404). All sensors should have phases that are roughly synchronized. Then a power spectrum can be measured, as a square of amplitude of the frequency domain signal (or a plot thereof) (step 406). Lastly, one or more sensors proximate to a given vessel can be identified from analysis of the power spectrum (step 408).

Cuff Application Verification and/or Correction

Another aspect of the invention generally concerns verifying and/or correcting the application (i.e., physical positioning and orientation) of the cuff assembly 32 (or any other embodiment of a cuff assembly) relative to the patient's limb L. In order for the system 30 to operate with relatively high accuracy and sensitivity, acoustic sensor elements must generally be located near the particular blood vessels of interest. As discussed above, the cuff assembly 32 can include one or more visible markers (e.g., markings 190 and 192) so that sensor elements in the array 58 are registered with respect to this icon located on the exterior of the cuff, and so that a user can nominally provide desired orientation of the cuff assembly 32 by orienting the markings. However, it may be desired to increase system resiliency by further decreasing operator dependencies, for instance, where an operator has limited knowledge of physiology or where, regardless of operator skill, a given patient's physiology compromises the operator's ability to appreciate subcutaneous blood vessel locations.

An optional feature of the present invention involves what is termed the Cuff Application Wizard (CAW), which can help assure that the cuff assembly 32 is applied properly with sensor elements located at desired positions relative to the patient's limb L. FIG. 11 is a flow chart that illustrated one embodiment of a method for implementing the CAW. It should be noted that the method as illustrated in FIG. 11 is shown merely by way of example and not limitation. Persons of ordinary skill in the art will recognize that various illustrated steps can be performed concurrently, or in a different order than shown. Moreover, additional steps not specifically listed can also be performed as desired for particular applications, and illustrated steps can be omitted in some embodiments.

The CAW process can work as follows. The cuff assembly 32 can be applied to the patient's limb L with markings 190 and/or 192 facing in nominally designated orientations (step 500), such as with the markings 190 facing anteriorly with respect to the patient's limb L. The cuff bladder 56 can be inflated to super-systolic levels occlude blood vessel(s) in the patient's limb L (step 200), and then be substantially linearly deflated (step 206) while acoustic sensors in utilized sensor arrays 58 are sampled (step 208). When Korotkoff sounds present, the acoustic sensors can sense those signals and convey them to an A/D converter in a real time and non-real time process for collection (step 222). The various sensor signals are interrogated to generate a plot by sensor position (step 502) and to determine areas of peak amplitudes (step 308). In alternative embodiments, loci of relative peak signal amplitudes can be determined by other methods that do not require generating a plot as with step 502. In this way, as discussed above, a map can be created that indicates the locations of primary limb arteries based on signal amplitude and frequency content of the acoustic sensor array 58 sensor signals, which can allow the system to automatically register individual microphone elements with specific arteries, which is useful in case the user knowledge of surface anatomy is inadequate or in case there is an anatomic anomaly (e.g., a vessel located in an atypical location). The CAW can further assess whether the loci of relative maxima among the sensor signals are within acceptable ranges (step 504). By pre-designating sensors with sensor numbers there is an acceptable range of sensor numbers within which each Korotkoff sound signal (or other desired sound signal) should fall. If the maxima fall in acceptable ranges, the positioning of the cuff assembly 32 can be deemed correct (step 506). If the cuff assembly 32 is incorrectly placed relative to the patient's limb L, an appropriate signal can be generated such that the user/operator can be notified of incorrect positioning (step 508), such as using the operator interface 60. After notification, the operator can reposition the entire cuff assembly 32, or can reposition individual sensors or groups of sensors as permitted by the particular embodiment of the cuff assembly 32. For example, if a nominal orientation of the cuff assembly 32 based on provided markings 190 and 192 or indicator flags 158A-3 should position a given sensor element (e.g., sensor element #1) in an anterior region of the patient's ankle, it would be expected that a relative maximum signal amplitude would be found within a specified number of sensors around the given sensor if the cuff assembly 32 is positioned correctly. Moreover, or in the alternative, if cuff assembly 32 is positioned correctly in an example test, anterior signal and posterior signals are in proper (i.e., expected) relation to each other when referencing which sensor number they correspond with.

FIGS. 12A and 12B are schematic cross-sectional representations of the patient's limb L with an embodiment of the cuff assembly 32 applied in different orientations. In FIG. 12A, correct positioning of the cuff assembly 32 is illustrated at an ankle. A designated anterior limb sensor element 158_{AL} is positioned at or near the anterior region of the limb L, specifically at or near the anterior tibial artery, and a designated medial limb sensor element 158_{ML} is positioned at or near the medial region of the limb L, specifically at or near the posterior tibial artery. It is expected that the anterior tibial artery and the posterior tibial artery are located within a given range of positions relative to each other. For instance, the anterior tibial artery and the posterior tibial artery can be expected to relate to each other at approximately an angle β measured about a longitudinal axis of the limb L. In FIG. 12B, incorrect positioning of the cuff assembly 32 is illustrated at the ankle. The designated anterior limb sensor element 158_{AL} is incorrectly positioned at or near the medial region of the limb L, specifically at or near the posterior tibial artery, and the designated medial limb sensor element 158_{ML} is positioned at or near the posterior region of the limb L, away from both anterior tibial artery and the posterior tibial artery.

Based on knowledge of a length of a sensor array 58, sensor element spacing, location of relative signal strength maxima, etc., the angle β (about and perpendicular to the long axis center of the limb L between an antero-postero line through the lower extremity and a line defined by the center of the cross sectional area of the limb and the location of the nth acoustic sensor in the acoustic array) between blood vessels, such as the anterior and posterior tibial arteries, can be calculated. Calculation of the angle β can optionally be used as error check for correct vessel identification, in combination with or as an alternative to other error checking procedures.

There will be limb circumference differences from one patient to another. If the acoustic sensor (e.g., the sensor element #1) in the array 58 associated with the external markings 190 faces anteriorly, this sensor (and those near it) will certainly register the anterior tibial artery regardless of limb circumference based on nominal positioning of the cuff assembly 32 on the limb L (in this case a leg) by an operator using the markings 190 as a guide. Provided that there are numerous sensors in the array 58, the sensors in the array 58 medial to sensor #1 will certainly register data for the posterior tibial artery regardless of limb circumference. The difference, based on limb size, will be which sensor number in the array 58 will sense maximum volume/signal strength. When the limb L has a relatively large diameter, sensors of the array 58 relatively far away from sensor #1 will register the posterior tibial artery. When the limb L has a relatively smaller diameter, sensors in the array 58 relatively closer to sensor #1 will register the posterior tibial artery. So the dependency or relationship between limb circumference and the sensor array 58 is that for smaller limbs L, the posterior tibial artery-located sensors, for example, will be closer to sensor #1 then for the larger limb case. Because the posterior tibial artery is always located medial to the anterior tibial artery in a leg, their locations can always be identified in this fashion.

For example, equation 3 can be used to calculate the angle β:

$$β = \frac{π \cdot {S}_{a}}{N} ( \frac{2 n - 1}{{C}_{L}} ) \qquad ( 3 )$$

where n is the sensor in the array returning a relative maximum signal, N is the total number of sensors in the array 58, Sₐ is a total length of the sensor array 58, and C_{L} is a circumference of the limb L. According to equation 3, N and Sₐ are generally constants for a given cuff assembly 32, and the circumference C_{L} can be assessed for each patient. The calculated angle β can be compared to an expected value or range, which can be constant or can be indexed to desired physiological parameters such as the circumference C_{L}.

Oscillometric Screening and Compensation Techniques

As discussed above, the system 30 can utilize data fusion techniques that combine auscultatory, oscillometric, PPG, tonometric, and/or other testing modalities. With respect to oscillometric techniques, the present invention can provide methods of screening to assess testing modality reliability, as well as to compensate for physiological conditions, such as pseudohypertension (and similar physiological conditions), that may affect test data. These methods can be utilized in conjunction with the data fusion techniques described above, or can be implemented in purely oscillometric systems that do not utilize data fusion.

Aside from auscultation of Korotkoff sounds, return of flow based on either Doppler ultrasound or photo-plethysmography (i.e., PPG methods) can be used. Another primary (non-auscultatory) method for assessing blood pressure is known as oscillometry. Examples of oscillometric systems are described in commonly-assigned U.S. Pat. Nos. 7,214,192, 7,172,555 and 7,166,076. One significant advantage of oscillometry over Doppler or photo-plethysmography is that oscillometry can measure systolic and diastolic pressure whereas Doppler and photo-plethysmography can only assess systolic pressure. Further, without a minimum threshold of blood flow velocity, Doppler signals may not be appreciated in some patients. Whereas Doppler ultrasound identifies the return-of-flow after occlusion with a blood pressure cuff to measure systolic pressure, oscillometry extracts specific morphological features from the plethysmographic pulse waveform to assess both systolic and diastolic pressure.

During an oscillometric test, the cuff bladder 56 can be inflated to super-systolic levels and then pressure in the cuff can be slowly deflated (e.g., at about 2 mm Hg/sec). Small expansions and contractions of the limb under test can be converted to a time varying electrical signal in synchrony with the patient's heartbeat. The oscillometric signal produced from this arrangement is very similar in morphology to intra-arterial blood pressure. An envelope of the peak-to-trough amplitude of this pulse signal can be acquired and stored in the memory 38-1 of the system 30. When measuring arm brachial systolic pressure (for a typical subject), the amplitude of the envelope first rises as the blood pressure cuff air pressure decreases (cuff deflates). Eventually, this envelope peaks and then starts to become smaller. The peak amplitude of the envelope (corresponding to the peak amplitude of the pulse signal) is identified. This point has been found, empirically, to be the mean arterial pressure (MAP). Further, a threshold called the systolic fraction can be identified, which can be a fixed, empirically determined fraction of the peak envelope amplitude. The air pressure in the cuff that corresponds to this fraction of the peak envelope amplitude (before the cuff has deflated through the MAP point) identifies the systolic blood pressure. Also, a threshold called the diastolic fraction is identified, which can be a fixed, empirically determined fraction of the peak envelope amplitude. The air pressure in the cuff that corresponds to this fraction of the peak envelope amplitude (after the cuff has deflated through the MAP point) identifies the diastolic blood pressure. When this arrangement is used to measure lower extremity systolic pressure, it is often found (particularly in cases of subjects with vascular disease) that the envelope does not rise and decline to the same extent as for the arm. This is attributed to the greater stiffness (i.e., lower compliance) of arteries in the legs of those with arterial disease. Thus, in these cases, the peak of the pressure oscillation envelope is difficult to identify. Further, the systolic and diastolic fractions are different for these instances of lower compliance arteries and even for ankle versus arm.

FIG. 13A is a graph of an example oscillometric cuff pressure signal P_{C} over time, and FIG. 13B is a graph of a normalized oscillogram 600 (i.e., an envelope waveform of pulse oscillations in the cuff pressure signal over time) expressed as peak-to-trough amplitude versus cuff pressure, which is a function ƒ(P_{C}) of the cuff pressure signal P_{C}. Discussion of oscillometric data and normalization for an oscillogram can be found in commonly-assigned U.S. Pat. Nos. 7,214,192, 7,172,555 and 7,166,076.

The present invention can measure characteristics of the oscillogram 600 waveform morphology (referred to herein as “slope”) to determine if the waveform slope is great enough to permit the identification of a distinct peak. In other words, a determination can be made as to the relative prominence of a peak in the oscillogram 600 waveform morphology relative to a substantially flat waveform. An empirically-derived threshold or range referred to as the critical systolic slope index (CSSI) can be used to assess slope, peak morphology, etc. If a peak is present (i.e., CSSI is above a given threshold or range), oscillometric testing can proceed in a normal fashion. If no peak is detectable (because the slope is less than a given threshold or range for the CSSI), then the oscillometric signal is determined to be insufficient to measure the blood pressure and, in an embodiment utilizing data fusion of multiple testing modalities, one of the remaining non-oscillometric modalities (i.e. photo-plethysmograph or auscultatory methods) can be used to determine blood pressure with the oscillometric modality playing no role in blood pressure measurement (or simply less highly weighted or contraindicated to a user). Further, if a peak is present but is relatively flat (such as falling within a particular range of CSSI values), compensation can be undertaken to provide more desirable testing data than would typically be provided. For instance, if the slope is greater than the CSSI but less than brachial systolic slope (i.e., slope found when measuring arm brachial pressure), then a compensatory factor can be applied to systolic and diastolic fractions to correct for deviation between behavior of the system when measuring arm pressure and the behavior when measuring ankle or other lower extremity limb pressure. Such compensation is described below.

For a patient with calcified or otherwise hardened arteries, testing may tend to produce a falsely elevated blood pressure value. Using an empirically-derived algorithm (i.e., mathematical formula), a pseudohypertension compensator can use a “raw” (i.e., unadjusted) pressure value along with pulse wave velocity (PWV) to produce an adjusted pressure value more reflective of intra-arterial blood pressure and less reflective of arterial wall stiffness. By way of example, one possible mathematical basis for the algorithm is described in reference to equations 4-16:

$$\begin{matrix}f ( Pc ) = \frac{1}{σ \sqrt{2 Π}} exp [ \frac{- {( Pc - MAP )}^{2}}{2 {σ}^{2}} ] ( normal distribution ) & ( 4 ) \\ Peak = f ( MAP ) = \frac{1}{σ \sqrt{2 Π}} γ & ( 5 ) \\ Psys - α s * Peak = \frac{α s}{σ \sqrt{2 Π}} & ( 6 ) \\ 2 * σ = MAP - {P}_{I} & ( 7 ) \\ σ = ( MAP - {P}_{I} ) / 2 & ( 8 ) \\ f ( Pc ) = \frac{1}{σ \sqrt{2 Π}} exp \frac{- {( Pc - MAP )}^{2}}{2 {σ}^{2}} & ( 9 ) \\ f ( Psys ) = \frac{α s}{σ \sqrt{2 Π}} = \frac{1}{σ \sqrt{2 Π}} exp [ \frac{- {( Psys - MAP )}^{2}}{2 {σ}^{2}} ] & ( 10 ) \\ α s = exp [ - {( Psys - MAP )}^{2} / 2 {σ}^{2} ] & ( 11 ) \\ ln [ α s ] = - [ \frac{{( Psys - MAP )}^{2}}{2 {σ}^{2}} ] & ( 12 ) \\ - 2 {σ}^{2} ln [ α s ] = {( Psys - MAP )}^{2} & ( 13 ) \\ Psys - MAP = \sqrt{- 2 {σ}^{2} ln [ α s ]} & ( 14 ) \\ Psys = MAP + \sqrt{- 2 {σ}^{2} ln [ α s ]} & ( 15 ) \\ Psys = MAP + \sqrt{- {2 [ \frac{MAP - PI}{2} ]}^{2} ln [ α s ]} & ( 16 )\end{matrix}$$

where P_{C} is cuff pressure, MAP is mean arterial pressure, P_{Sys} is systolic pressure, P_{I} is inflation start pressure, P_{D} is deflation start pressure, and xs is a fraction of the Peak corresponding to systolic pressure (P_{sys}). Equations 4-16 above relate systolic pressure to mean arterial pressure (MAP), waveform standard deviation and αs.

As arterial stiffness increases, due to pseudohypertension or other physiologic factors, the oscillogram 600 shown in FIG. 13B shifts to the right (i.e., increases in pressure) and values such MAP shift to the right (i.e., increase in pressure). As shown in FIG. 13B, an example shifted oscillogram 602 is illustrated, which represents a rightward shift from the oscillogram 600 (which may in turn be shifted from a healthy nominal waveform, not shown). Values associated with the oscillogram 600 are designated with a subscript “0” and values associated with the oscillogram 602 are designated with a subscript “1”.

A compensation factor γ can be empirically derived to adjust for blood vessel stiffness. For instance, the compensation factor γ can be established with γ=1 for a normal, healthy vessel and γ>1 for increased arterial stiffness. The compensation factor can compare arm versus leg values, or be derived in another suitable manner. The exact value of γ used for a particular compensation can be selected as desired as a function of arterial stiffness, that is, based on the degree of arterial stiffness of a given patient the value of the compensation factor γ can vary accordingly. For instance, a table of values or tiered system of values can be developed, etc. In one embodiment, possible values of γ can each be associated with various CSSI values. Using the compensation factor γ, values such as MPA and P_{Sys} can be derived from sensed data using equations 17 and 18 or 19:

$$\begin{matrix}MAP = γ {MAP}_{0} & ( 17 ) \\ Psys ( adj ) = γ {MAP}_{0} + \sqrt{- 2 {σ}^{2} ln [ α s ]} \\ OR & ( 18 ) \\ Psys ( adj ) = γ {MAP}_{0} + \sqrt{- {2 [ \sqrt{\frac{γ {MAP}_{0} - {P}_{I}}{2}} ]}^{2} ln [ α s ]} & ( 19 )\end{matrix}$$

The following equations illustrate one example. If MAP₀=93 mmHg and σ=70 mmHg for a healthy vessel with γ=1, then

$$Psys = 1 ( 93 ) + \sqrt{- {2 [ \frac{70}{2} ]}^{2} ln ( 0.6 ) mm Hg}$$

$$Psys ≅ 128.4 mm Hg$$

Whereas for a vessel with increased arterial stiffness having MAP₀=93 mmHg, σ=70 mmHg and γ=1.2, then an adjusted systolic pressure value P_{Sys}(adj) is given by

Psys(adj)=93(1.2)+35.4≠147 mmHg

Thus, with increasing arterial stiffness, the oscillogram 600 shifts to the right and Psys is modified upwards.

In addition or in the alternative, changes in the oscillogram 600 envelope morphology can be modeled leading to changes in a (standard deviation) as a compensation factor, which can vary as a function of patient physiology.

The well-known Moens-Korteweg formula can relate pulse wave web of blood flow to arterial stiffness, given by equation 20:

$$PWV = k \sqrt{\frac{Eh}{2 ργ}} \\ where \\ E = \frac{{σ}_{t}}{{E}_{t}} = Young ’ s Modulus of Elasticity \\ stress = {σ}_{t} = \frac{Pr}{h} \\ {E}_{t} = \frac{dr}{r} = strain \\ r = vessel radius \\ P = pressure \\ h = vessel wall thickness \\ ρ = rho ( density of blood ) \approx 1.03 to 1.07 g \text{/} ml ( e . g . , 1.05 g \text{/} ml ) \\ k = an empirically \text{-} derived constant \qquad ( 20 )$$

Therefore, as derived from equation 19:

PWV=k\sqrt{γ} [symbol] γ=k(PWV)²

Psys(adj)=k(PWV)²+\sqrt{−2σ² ln(αs)}

Concluding Remarks

In view of the entire present disclosure, persons of ordinary skill in that art will recognize that the present invention provides numerous advantages and benefits. For example, the auscultatory approach of the present invention can produce test values similar to Doppler approaches without the complexity associated with the use of ultrasound. Additionally, prior art Doppler-based methods may suffer from a time delay disadvantage relative to microphone-based (i.e., auscultatory) methods, related to the distance between that occlusive cuff and a Doppler probe positioned distally from the site of occlusion that senses return of blood flow, which will often result inartificially lower pressure values than an auscultatory approach. This time delay (which is equal to the distance between cuff and probe divided by the velocity of blood) is much higher (approximately 10×) than the time for the pulse wave to travel from cuff to microphone (for a microphone positioned in the cuff).

Any relative terms or terms of degree used herein, such as “substantially”, “essentially”, “generally” and the like, should be interpreted in accordance with and subject to any applicable definitions or limits expressly stated herein. In all instances, any relative terms or terms of degree used herein should be interpreted to broadly encompass any relevant disclosed embodiments as well as such ranges or variations as would be understood by a person of ordinary skill in the art in view of the entirety of the present disclosure, such as to encompass ordinary manufacturing tolerance variations, incidental alignment variations, incidental and background noise, and the like.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims. Teachings of any described embodiment can generally be combined with teachings of any other embodiment, as desired. For example, acoustic sensors can be placed on a slider band within a cuff in combination with tabs or a transparent window, as described above with respect to various embodiments. Moreover, methods described with respect to one embodiment of associated equipment can generally be performed using any other embodiment or configuration of the relevant equipment, unless specifically noted.

1. A vascular testing system comprising:
a first pressure cuff assembly positionable about a limb of a patient, the first pressure cuff assembly comprising:
a pressure bladder; and
a first array of acoustic sensors, the first array comprising a plurality of acoustic sensor elements arranged circumferentially relative to the pressure bladder; and
a controller configured to concurrently sense vascular data at two or more discrete testing locations utilizing at least two different sets of the acoustic sensors of the first array each positioned at or near one of the discrete testing locations, wherein each set of the acoustic sensors includes one or more of the plurality of acoustic sensor elements.

2. The system of claim 1, the first pressure cuff assembly further comprising:
a second array of acoustic sensors, the second array comprising a plurality of acoustic sensor elements arranged circumferentially relative to the pressure bladder, wherein the second array is axially spaced from the first array.

3. The system of claim 1 and further comprising:
a second pressure cuff assembly configured to be secured about a different limb of the patient than the first pressure cuff, the second pressure cuff assembly comprising:
a pressure bladder; and
a first array of acoustic sensors, the first array comprising a plurality of acoustic sensor elements arranged circumferentially relative to the pressure bladder.

4. The system of claim 1, wherein at least a portion of the acoustic sensor elements of the first array are independently repositionable relative to each other.

5. The system of claim 1, the first pressure cuff assembly further comprising:
an attachment mechanism for adjustably securing at least one of the acoustic sensor elements of the first array to a body of the first pressure cuff assembly.

6. The system of claim 5, wherein the attachment mechanism is selected from the group consisting of hook-and-loop structures and a clip.

7. The system of claim 1, the first pressure cuff assembly further comprising:
a cuff body relative to which the cuff bladder is supported; and
a band to which the first array of acoustic sensors is supported, the band and the first array of acoustic sensors configured to be repositionable relative to the cuff body.

8. The system of claim 1, the first pressure cuff assembly further comprising:
a cuff body relative to which the cuff bladder is supported;
an at least partially transparent or translucent window in the cuff body configured such that at least a portion of the first array of acoustic sensors is visible through the window.

9. A method of vascular testing comprising:
positioning a pressure bladder about a limb of a patient;
sensing pressure associated with the pressure bladder;
positioning a first circumferential array of acoustic sensors about the limb of the patient at or near the pressure bladder;
sensing vascular data at a first testing location utilizing a first set of acoustic sensors of the first circumferential array; and
sensing vascular data at a second testing location utilizing a second set of acoustic sensors of the first circumferential array that is different from the first set of acoustic sensors, wherein the vascular data is sensed at both the first and second testing locations concurrently.

10. The method of claim 9, wherein the step of sensing vascular data at a first testing location utilizing a first set of acoustic sensors of the first circumferential array comprising sensing vascular data for an anterior tibial artery, and wherein the step of sensing vascular data at a second testing location utilizing a second set of acoustic sensors of the first circumferential array that is different from the first set of acoustic sensors comprises sensing vascular data for an posterior tibial artery.

11. The method of claim 9 and further comprising:
associating the first set of acoustic sensors with data for a first blood vessel; and
associating the second set of acoustic sensors with data for a second blood vessel.

12. The method of claim 9 and further comprising:
identifying a first relative maximum as a function of acoustic signals of the sensors of the first circumferential array; and
associating the first set of acoustic sensors with data for a first blood vessel as a function of the first relative maximum.

13. The method of claim 12 and further comprising:
identifying a second relative maximum as a function of acoustic signals of the sensors of the first circumferential array; and
associating the second set of acoustic sensors with data for a second blood vessel as a function of the second relative maximum.

14. The method of claim 12 and further comprising:
determining whether the first relative maximum is located within an expected range of sensor elements within the array of acoustic sensors; and
generating a notification signal if the first relative maximum is not located within an expected range of sensor elements within the array of acoustic sensors.

15. The method of claim 14 and further comprising:
determining whether the second relative maximum is located within an expected range of sensor elements within the array of acoustic sensors.

16. The method of claim 14 and further comprising:
repositioning at least one sensor element of the first circumferential array of acoustic sensors; and
sensing vascular data at the first testing location utilizing the first circumferential array of acoustic sensors as repositioned.

17. The method of claim 9 and further comprising:
screening the vascular data sensed at the first testing location as a function of vessel stiffness.

18. The method of claim 17, wherein the step of screening the vascular data sensed at the first testing location as a function of vessel stiffness comprises analyzing data obtained using another testing modality.

19. The method of claim 9 and further comprising:
screening the vascular data sensed at the first testing location as a function of pulse wave velocity for a physiological factor that influences resultant test data.

20. The method of claim 19, wherein the step of screening the vascular data sensed at the first testing location as a function of pulse wave velocity for a physiological factor that influences resultant test data further comprises:
positioning a second circumferential array of acoustic sensors about a limb of a patient at or near the pressure bladder and axially spaced from the first circumferential array of acoustic sensors; and
sensing vascular data at a distal portion of the first testing location utilizing a first set of acoustic sensors of the second circumferential array.

21. The method of claim 9 and further comprising:
obtaining test data utilizing another testing modality; and
fusing data obtained using data from at least the first set of acoustic sensors of the first circumferential array and the test data of the other testing modality.

22. The method of claim 21 and further comprising:
sensing pressure oscillations in a cuff positioned about a limb;
comparing characteristics of sensed pressure oscillation waveform morphology data in relation to a critical systolic slope index; and
adjusting sensed data as a function of a compensation factor where the critical systolic slope index is within a given range.

23. A method of auscultatory vascular testing comprising:
positioning an array of acoustic sensors about a limb of a patient;
concurrently sensing data with the array of acoustic sensors; and
selecting sensed data outputs of at least two circumferentially spaced sensors of the array of acoustic sensors as representative of respective physiological conditions of a first blood vessel and a second blood vessel of the limb.

24. The method of claim 23 and further comprising:
validating positioning of the array of acoustic sensors relative to the limb as a function of the sensed data.

25. The method of claim 23 and further comprising:
positioning a pressure bladder about the limb, wherein the array of acoustic sensors is positioned at or near the pressure bladder.

26. A method of vascular testing comprising:
sensing pressure oscillations in a cuff positioned about a limb;
comparing characteristics of sensed pressure oscillation waveform morphology data in relation to a critical systolic slope index; and
adjusting sensed data as a function of a compensation factor where the critical systolic slope index is within a given range.
